(19) United States
(12) Patent Application Publication
(10) Pub. No.: US 2024/0156742 A1
(43) Pub. Date: May 16, 2024

(54) TARGETED MULTIFUNCTIONAL NANOSTRUCTURED LIPID CARRIERS

(71) Applicant: TECHNION RESEARCH & DEVELOPMENT FOUNDATION LIMITED, Haifa (IL)

(72) Inventors: Yehuda G. ASSARAF, Haifa (IL); Yoav D. LIVNEY, Haifa (IL); Lital COHEN, Haifa (IL)

(21) Appl. No.: 18/407,721

(22) Filed: Jan. 9, 2024

Related U.S. Application Data
(63) Continuation of application No. PCT/IL2022/051057, filed on Oct. 3, 2022.
(60) Provisional application No. 63/251,750, filed on Oct. 4, 2021.

(51) Int. Cl. A61K 9/51

(52) U.S. Cl. CPC A61K 9/5123; A61K 9/5146

(57) ABSTRACT

This invention, in some embodiments thereof, provides a core-shell nanoparticle including a lipid-PSMA conjugate, encapsulating a liquid oil-based core. The invention further provides aqueous compositions including the core-shell nanoparticles, and methods for using same, such as for the treatment of proliferative diseases associated with PSMA upregulated expression.

### CROSS REFERENCE TO RELATED APPLICATIONS

This application is a Bypass Continuation of PCT Patent Application No. PCT/IL2022/051057, having International filing date of Oct. 3, 2022, which claims the benefit of priority of U.S. Provisional Patent Application No. 63/251,750 filed Oct. 4, 2021, entitled “DEVELOPMENT OF NOVEL SELECTIVELY TARGETED MULTIFUNCTIONAL NANOSTRUCTURED LIPID CARRIERS FOR PROSTATE CANCER TREATMENT”, the contents of which are all incorporated herein by reference in their entirety.

### FIELD OF INVENTION

This invention is directed to, nanoparticles comprising a PSMA ligand and encapsulating an active agent.

### BACKGROUND OF THE INVENTION

Cancer is one of the leading causes of morbidity and mortality worldwide. In this respect, prostate cancer (PC) is the most common cancer in men over the age of 50 and the 4th most prevalent human malignancy. The current modalities to treat early-stage PC include surgery, radiation therapy, and for some men, androgen deprivation therapy (ADT). The treatment of PC with ADT is based upon the crucial function of the androgen receptor (AR), which drives an intricate regulation of expression of AR-responsive genes. AR is a member of the nuclear receptor superfamily with a similar structure to the estrogen receptor, progesterone receptor, and thyroid hormone receptor. Testosterone and dihydrotestosterone bind to the ligand-binding domain (LBD) of AR, resulting in conformational change and translocation of AR into the nucleolus. Therein, AR forms a dimer and binds to the androgen-response element (ARE) of the promoter and the enhancer of targeted genes through the zinc-finger of the AR DNA-binding domain. The formation of the AR coactivator complex enhances the transcriptional activity of androgen-regulated genes (ARGs). Previous studies have shown a network of ARGs that supports the development, progression, and metastasis of PC. Unfortunately, however, PC cells frequently acquire resistance to hormonal therapy, resulting in local relapse, progression, and metastasis. This latter phase of the disease is referred to as castration-resistant prostate cancer (CRPC) or metastatic CRPC (mCRPC), which can further become a lethal disease. The most common metastases are lymph node and bone metastases. The current treatment of CRPC includes systemic second-generation endocrine treatment, followed by combination chemotherapy, where docetaxel (DTX) and cabazitaxel (CTX) are the cytotoxic drugs approved for the treatment of this stage of the disease. Like the parent drug paclitaxel (PTX), DTX and CTX are taxanes that promote microtubule assembly and inhibit microtubule depolymerization, thereby causing an antimitotic cytotoxic effect. This treatment is limited by low drug bioavailability due to the poor solubility of these therapeutic agents in the blood and the side effects to healthy tissues, including osteoporosis, insulin resistance, and reduced quality of life. Furthermore, while ADT exhibits a significant therapeutic benefit, its effectiveness is transient, as PC cells develop molecular mechanisms which allow them to survive and proliferate even under testosterone deprivation. The goal of cancer therapeutics is to increase the survival time and the quality of life of the patient by reducing systemic toxicity.

### SUMMARY OF THE INVENTION

In one aspect of the invention, there is provided a conjugate comprising a fatty acid derivative covalently bound to a prostate specific membrane antigen (PSMA) ligand, wherein the covalently bound is via a linker; wherein the PSMA ligand is represented by Formula 1:

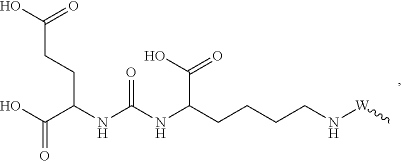

including any salt, and any stereoisomer thereof; wherein W is a spacer, or is absent; and wherein the wavy bond represents an attachment point to the liker.

In one embodiment, W is absent, and wherein a first portion of the linker is covalently bound to a carbonyl group of the fatty acid derivative , and a second portion of the linker is covalently bound to an c-amine of the lysine of the PSMA ligand.

In one embodiment, the fatty acid derivative is a C10-C30 fatty acid derivative.

In one embodiment, the linker comprises PEG.

In one embodiment, the linker has a molecular weight (MW) between 1,000 and 5,000 Dalton (Da).

In one embodiment, the c-amine of the lysine is bound to the linker via an amide bond.

In one embodiment, the conjugate is represented by Formula 2:

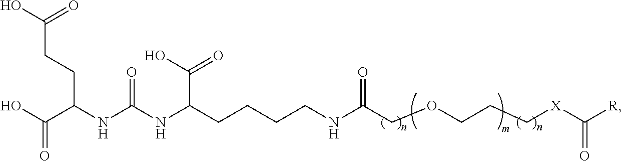

including any salt, and any stereoisomer thereof; wherein R is selected from a C10-C30 alkyl, a C10-C30 alkenyl, and a C10-C30 alkynyl; X represents O, NH, or S; each n is independently 0, or between 1 and 10; and m is between 2 and 100.

In another aspect, there is provided a composition comprising a plurality of nanoparticles, wherein each of the plurality of nanoparticles comprises a core and a shell, wherein: the shell comprises the conjugate of the invention; and the core comprises a liquid oil.

In one embodiment, the composition further comprising a PEG-ylated fatty acid.

In one embodiment, the composition further comprising an active agent.

In one embodiment, the active agent is an anti-cancer agent; and wherein the anti-cancer agent is an oil-soluble compound.

In one embodiment, the plurality of nanoparticles is characterized by an average particle size in a range of between about 30 and about 300 nm.

In one embodiment, the plurality of nanoparticles is characterized by a negative zeta potential between 0.5 and 50 mV.

In one embodiment, a weight ratio between the liquid oil and the PEG-ylated fatty acid is between about 1:1 and about 1:3.

In one embodiment, the average particle size is between about 50 and about 200 nm, and wherein a molar ratio between the active agent and the PEG-ylated fatty acid is between about 0.1:1 and about 1:1.

In one embodiment, the liquid oil is a fatty acid.

In one embodiment, the composition further comprising an aqueous solution, wherein the plurality of nanoparticles is dispersed within the aqueous solution.

In another aspect there is provided a pharmaceutical composition comprising the composition of the invention, and a pharmaceutically acceptable carrier.

In one embodiment, the pharmaceutical composition is formulated for systemic or local administration.

In one embodiment, the pharmaceutical composition is for use in treatment of a PSMA-related disease or disorder in a subject in need thereof.

In another aspect, there is provided a method for introducing an active agent into a PSMA-expressing cell, the method comprising contacting the cell with an effective amount of the composition of the invention, and the agent, wherein the agent is encapsulated within the plurality of nanoparticles, thereby introducing the active agent into a PSMA-expressing cell.

In one embodiment, the cell is a prostate gland cell.

In one embodiment, the cell is a prostate cancer cell.

In one embodiment, the active agent is an oil-soluble compound.

In one embodiment, the active agent in an anti-cancer agent.

In one embodiment, the cell is obtained or derived from a subject afflicted with prostate cancer.

In another aspect, there is provided a method for treating a PSMA-related disease or disorder in a subject in need thereof, the method comprising administering to the subject a therapeutically effective amount of the pharmaceutical composition of the invention, thereby treating the PSMA-related disease or disorder in a subject.

In one embodiment, the PSMA-related disease or disorder is a cell-proliferation related disease.

In one embodiment, the cell-proliferation related disease is cancer.

In one embodiment, the cancer is prostate cancer.

In one embodiment, at least one cell of the prostate cancer is characterized by increased expression, abundance, or both, of PSMA transcript, protein product thereof, or both, compared to a control.

In one embodiment, the method further comprising a step preceding the administering, comprising determining expression, abundance, or both, of PSMA transcript, protein product thereof, or both, in a sample derived or obtained from the subject, wherein an expression, abundance, or both, of the PSMA transcript, protein product thereof, or both, being above a predetermined threshold, is indicative of the being suitable for the administering.

In one embodiment, the sample comprises at least one cell of the prostate cancer.

In one embodiment, the treating comprises: reducing the number of proliferating cells of the cancer, reducing the rate of cell proliferation of cells of the cancer, reducing the survival or viability of cells of the cancer, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 includes a non-limiting scheme showing prostate cancer (PC) progression and treatment modalities.

FIG. 2 includes a non-limiting illustration showing common cell surface antigens used for active PC targeting via selective receptor-mediated endocytosis. One of the most highly selective and well-characterized biomarker antigens of PC is the PSMA receptor. Various targeting moieties were found to have specific interactions with the PSMA receptor, including aptamers and antibodies.

FIG. 3 includes synthesis scheme showing the synthesis of SA-PEG with PSMA TL Glu-Urea-Lys. SA-PEG-COOH was conjugated to Glu-Urea-Lys with protecting groups of tert-Butyl esters. The SA-PEG-TL was subjected to acidic conditions for the removal of the protecting groups, hence regaining the carboxylic groups of the PSMA TL.

FIG. 4 includes a vertical bar graph showing mean diameter of monomodal distribution of nanostructured lipid carriers (NLCs) with various liquid lipids. NLCs formulation of 60% SA-PEG and 40% of different liquid lipids were examined for size distribution. Values presented are means±standard error (SE), n=2.

FIG. 5 includes a vertical bar graph showing mean diameter of monomodal distribution of Enzalutamide (ENZ)-loaded NLCs. NLCs formulation of 60% SA-PEG and 40% liquid lipids loaded with ENZ in 0.4:1 ENZ:SA-PEG molar ratio was examined for size distribution. Values presented are means±SE, n=2.

FIG. 6 includes spectra of H-NMR spectroscopy. Graphs represent SA-PEG-TL with tert-Butyl esters protecting groups (upper spectrum) compared to the final conjugation product SA-PEG-TL (lower spectrum). The H-NMR spectroscopy confirms that the tert-Butyl ester protecting groups were removed, thus revealing the carboxylic groups of the TL.

FIG. 7 includes a vertical bar graph showing mean diameter and zeta-potential of nanoparticles (NPs) at increasing Cabazitaxel (CTX):SA-PEG molar ratios. Columns represent the mean diameter and black diamonds represent zeta-potential. Values presented are means±SE, n=3.

FIG. 8 includes a vertical bar graph showing mean diameter and zeta-potential of NPs at increasing ENZ:SA-PEG molar ratios. Columns represent the mean diameter and black diamonds represent zeta-potential. Values shown are means±SE, n=3.

FIG. 9 includes a vertical bar graph showing CTX loading capacity (LC) and encapsulation efficiency (EE) at increasing CTX:SA-PEG molar ratios. Columns represent the LC whereas circles denote EE. Values shown are means±SE, n=3.

FIG. 10 includes a vertical bar graph showing ENZ loading capacity (LC) and encapsulation efficiency (EE) at increasing ENZ:SA-PEG molar ratios. Columns represent the LC and circles denote EE. Values shown are means±SE, n=3.

FIGS. 11A-11C include micrographs showing analysis of NPs morphology by Cryo-TEM. Cryo-TEM images of (11A) 0.6:1 CTX:SA-PEG NPs; (11B) 0.4:1 ENZ:SA-PEG NPs; and (11C) non-encapsulated NLCs in water. NPs size was analyzed using ImageJ software.

FIG. 12 includes a graph showing kinetics of CTX drug release. In vitro drug release profiles of CTX-loaded NPs (circles) compared to unencapsulated CTX (triangles). Values shown are means±SE, n=2.

FIG. 13 includes a graphs showing kinetics of ENZ drug release. In vitro drug release profiles of ENZ-loaded NPs (circles) compared to unencapsulated ENZ (triangles). Values shown are means±SE, n=2.

FIGS. 14A-14B include micrographs showing immunohistochemistry analysis of PSMA expression. PSMA expression (labeled in brown) was examined in LNCaP (14A) and PC-3 (14B) cell lines by the department of pathology in Rambam Health Care Campus.

FIGS. 15A-15D include fluorescent micrographs showing selective targeting of NPs decorated with increasing concentrations of the PSMA TL. Confocal laser microscopy images of LNCaP cells, following 2 hr incubation with: (15A) NPs lacking TL decoration; (15B) NPs decorated with 15 nM; (15C) 30 nM, and (15D) 80 nM of TL (at 37° C.; samples were diluted 1:400 (v/v) in FBS-free medium). Nuclear DNA staining was achieved with Hoechst 33342 (1 μg/ml) and is marked in blue, whereas NPs labeled with Cy7 are marked in red.

FIGS. 16A-16F include fluorescent micrographs showing specific internalization of targeted NPs. Confocal microscopy images of LNCaP (16A), PC-3 (16B), 1975 (16C), HEK293 (16D), BEAS2B (16E), and FSE (16F) cells, following 2 hr incubation with targeted NPs labelled with Cy7 (at 37° C.; Samples were diluted 1:400 (v/v) in FBS-free medium). Nuclear DNA staining achieved with Hoechst 33342 (1 μg/ml) is marked in blue, whereas NPs labelled with Cy7 are marked in red.

FIGS. 17A-17B include fluorescent micrographs showing characterization of active internalization of targeted NPs to LNCaP cells. Confocal laser microscopy images of LNCaP cells, following 1 hr incubation with targeted NPs labeled with Cy7 (samples were diluted 1:400 (v/v) in FBS-free medium) at two different temperatures: 37° C. (17A) and 4° C. (17B). Nuclear DNA staining achieved with Hoechst 33342 (1 μg/ml) is marked in blue, whereas NPs labeled with Cy7 are marked in red.

FIG. 18 includes a graph showing selective growth inhibition of CTX-loaded NPs to LNCaP target cells. Cell growth inhibition as a function of CTX concentration encapsulated within NPs (0.6:1 CTX:SA-PEG molar ratio) in LNCaP target cells (circles) and PC-3 non-target cells (triangle). Values presented are means±SE. The sigmoidal model curve of LNCaP cells was fitted using Equation (3).

FIG. 19 includes a graph showing inhibitory effect of free CTX. Cell viability as a function of free CTX concentration of LNCaP target cells (circles) and PC-3 non-target cells (triangle). Values presented are means±SE. Sigmoidal model curves were fitted using Equation (3).

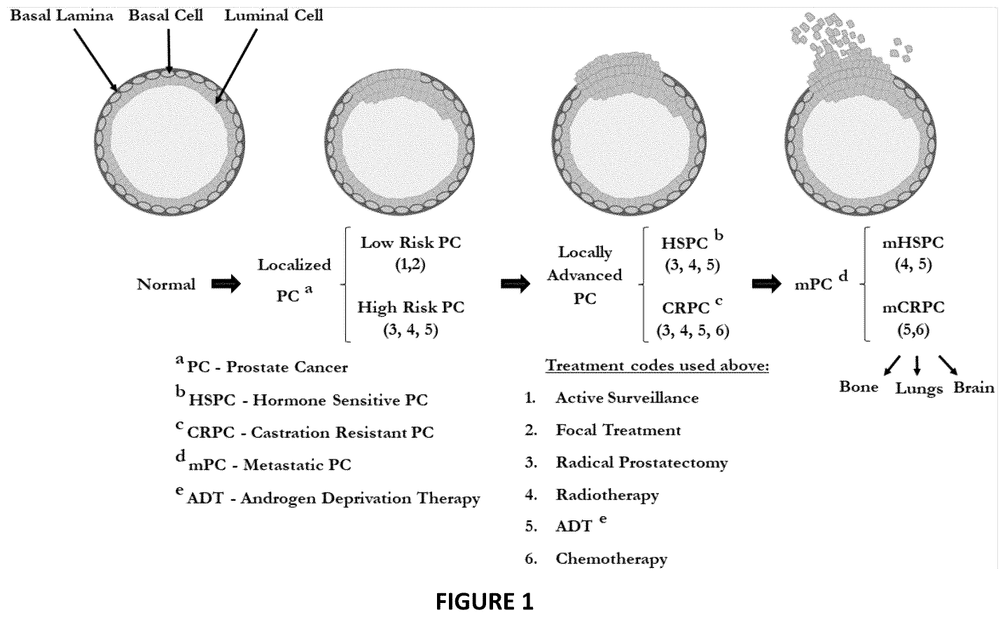

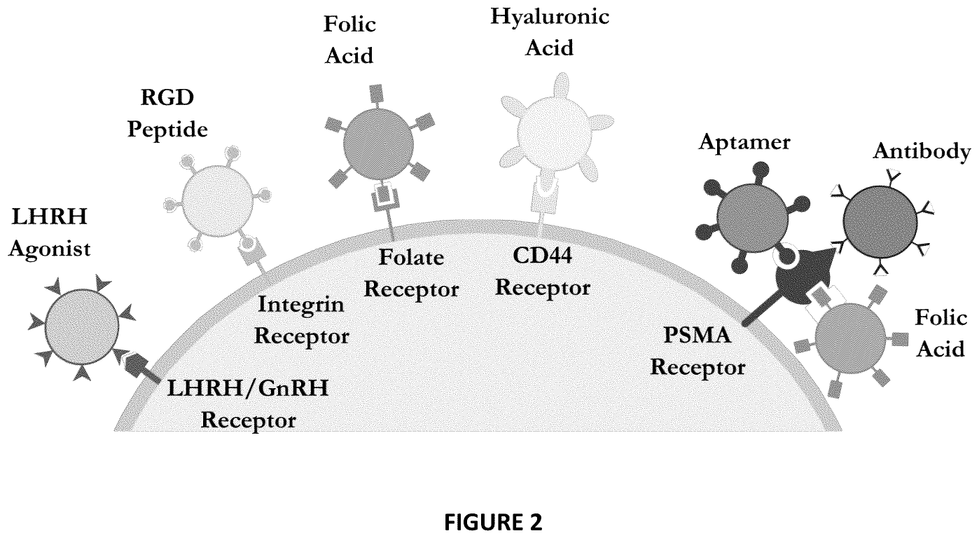

### DETAILED DESCRIPTION OF THE INVENTION

In one embodiment, the present invention provides a conjugate comprising a fatty acid derivative covalently bound to a Prostate Specific Membrane Antigen (PSMA) ligand. In some embodiments, the conjugate of the invention is water-soluble or a water-dispersible compound.

In some embodiments, the conjugate of the invention comprises a fatty acid derivative covalently bound to the PSMA ligand via a linker. In some embodiments, the PSMA ligand is characterized by a binding affinity to the PSMA receptor. In some embodiments, the ligand is a peptide. In some embodiments, the ligand binds PSMA on the cell membrane. In some embodiments, the ligand comprises a single specie or a plurality of chemically distinct species.

In some embodiments, the PSMA ligand hybridizes to the PSMA receptor. In some embodiments, the PSMA ligand is complementary to the PSMA receptor. The structures of various PSMA ligands are well known, and include a Glu-Lys dipeptide having a urea linkage between the amino acids. The PSMA receptor may further comprise a spacer (e.g. a small molecule such as a natural and/or unnatural amino acid, alkyl, an aliphatic ring, an aromatic ring, a heteroaromatic ring; a bond, such as an amide bond, an ester bond, a thioester bond, a disulfide bond, including any combination thereof). Exemplary PSMA ligands can be derived (by the obstruction of the F-18 atom, and or fluoro-pyridine/fluorobenzyl moiety) from the known F18-labeled PET tracers, such as 18F-DCFBC, 18F-DCFPyL, 18F-PSMA-1007, etc.

In some embodiments, the PSMA ligand further comprises a chelating moiety (e.g. DOTA, NOTA, NODAGA, HBED-CC, etc.).

In some embodiments, the PSMA ligand is represented by Formula 1:

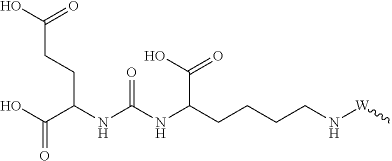

including any salt, and any stereoisomer thereof; wherein W is a spacer, a chelating moiety or is absent; and wherein the wavy bond represents an attachment point to the liker.

In some embodiments, the conjugate of the invention comprises a single PSMA ligand. In some embodiments, the conjugate of the invention comprises a single fatty acid derivative.

In some embodiments, the spacer has a MW less than 1,000 Da, less than 900 Da, less than 800 Da, less than 700 Da, less than 600 Da, less than 500 Da, less than 400 Da, less than 300 Da, less than 200 Da, less than 100 Da. Each possibility represents a separate embodiment.

In some embodiments, the spacer is absent and the PSMA ligand is bound to the linker via the ε-amine of the lysine:

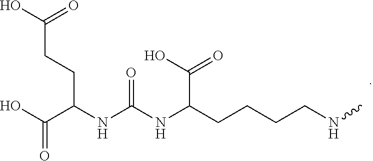

In some embodiments, the fatty acid derivative is a mono-carboxylic acid derivative. In some embodiments, the fatty acid derivative is represented by Formula:

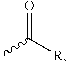

wherein R represents a C5-C30 alkyl, a C5-C30 alkenyl, or a C5-C30 alkynyl, wherein each of the alkyl, alkenyl and alkynyl is optionally substituted; and wherein the wavy bond represents an attachment point to the liker.

In some embodiments, R represents a C9-C29 alkyl, a C9-C29 alkenyl, or a C9-C29 alkynyl. In some embodiments, R represents a C9-C19 alkyl, a C9-C19 alkenyl, or a C9-C19 alkynyl. In some embodiments, R represents a C15-C19 alkyl, a C15-C19 alkenyl, or a C15-C19 alkynyl.

In some embodiments, the conjugate is represented by Formula:

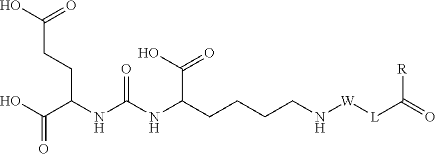

wherein, W and R are as described hereinabove, and wherein L represents the linker.

In some embodiments, the linker of the invention is or comprises a linear or a branched chain. In some embodiments, the linker of the invention is or comprises a backbone comprising a linear or a branched chain.

In some embodiments, the linker of the invention comprises a biocompatible polymer. In some embodiments, the biocompatible polymer is at least partially biodegradable.

In some embodiments, the linker comprises a polymer. In some embodiments, the backbone (e.g., a polymer chain) of the linker is covalently bound to fatty acid derivative and to the PSMA ligand. In some embodiments, the backbone of the linker comprises a first end covalently bound to the lipid and a second end covalently bound to the targeting moiety.

In some embodiments, the polymer is biocompatible. In some embodiments, the biocompatible polymer is a biodegradable polymer. In some embodiments, the biocompatible polymer comprises a polyether (e.g. polyglycol ether), a polyester, a polyamide, or any combination or a co-polymer thereof.

In some embodiments, the biocompatible polymer is selected from the group consisting of a polyether (e.g. PEG), a polyacrylate or an ester thereof, a polyacrylamide, a polyester (e.g., polylactide, polyglycolic), a polyanhydride, a polyvinyl alcohol, a polysaccharide, a poly(N-vinylpyrrolidone), a polyoxazoline, a poly(amino acid), or any combination or a co-polymer thereof. Each possibility represents a separate embodiment of the invention.

As used herein, the terms “peptide”, “polypeptide”, “polyamino acid” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide”, “polyamino acid” and “protein” apply to naturally occurring amino acid polymers including or consisting essentially of 21 naturally occurring amino acids. In one embodiment, the terms “peptide”, “polypeptide”, “polyamino acid” and “protein” apply to naturally occurring amino acid polymers including or consisting essentially of 21 naturally occurring amino acid residues bound to each other via alpha peptide bonds. In one embodiment, the terms “peptide”, “polypeptide”, “polyamino acid” and “protein” apply to naturally occurring amino acid polymers including or consisting essentially of 21 naturally occurring amino acid residues bound to each other via a primary amide bond. In one embodiment, the terms “peptide”, “polypeptide”, “polyamino acid” and “protein” apply to naturally occurring amino acid polymers including or consisting essentially of 21 naturally occurring amino acid residues bound to each other via a peptide bond formed by a formal condensation between alpha amino group of the first amino acid and alpha carboxy group of the next following amino acid. In one embodiment, the terms “peptide”, “polypeptide”, “polyamino acid” and “protein” apply to naturally occurring amino acid polymers including or consisting essentially of 21 naturally occurring amino acid residues bound to each other via a peptide bond formed by a formal condensation between (i) alpha amino group, or alpha carboxy group of the first amino acid, and (ii) a side chain amino group, or a side chain carboxy group of the next following amino acid. In one embodiment, the terms “peptide”, “polypeptide”, “polyamino acid” and “protein” apply to naturally occurring amino acid polymers including or consisting essentially of 21 naturally occurring amino acids. In another embodiment, the terms “peptide”, “polypeptide”, “polyamino acid” and “protein” apply to amino acid polymers in which one or more amino acid residue is an artificial chemical analogue of a corresponding naturally occurring amino acid.

The term “artificial chemical analogue” or “chemical derivative” includes any chemical derivative of the polypeptide having one or more residues chemically derivatized by reaction on the side chain or on any functional group within the peptide. Such derivatized molecules include, for example, peptides bearing one or more protecting groups (e.g., side chain protecting group(s) and/or N-terminus protecting groups), and/or peptides in which free amino groups have been derivatized to form amine hydrochlorides, p-toluene sulfonyl groups, carbobenzoxy groups, t-butyloxycarbonyl groups, acetyl groups or formyl groups. Free carboxyl groups may be derivatized to form amides thereof, salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and Dab, Daa, and/or ornithine (O) may be substituted for lysine.

In some embodiments, the biocompatible polymer is hydrophilic. In some embodiments, the biocompatible polymer is or comprises a polyether.

In some embodiments, the polyether comprises a backbone comprising a plurality of alkoxylate-based repeating units. In some embodiments, the polyether is represented by a general formula: —(RO)x-, wherein R represents C1-C10 alkyl; and x is an integer ranging between 2 and 1000. In some embodiments, R represents an alkyl comprising between 1 and 10, between 1 and 2, between 2 and 4, between 4 and 10, between 2 and 5, between 1 and 5, between 5 and 10 carbon atoms, including any range between. In some embodiments, x is between 2 and 1000, between 2 and 100, between 2 and 10, between 2 and 50, between 50 and 100, between 2 and 200, between 50 and 200, between 50 and 100, between 100 and 200, between 200 and 500, between 500 and 1000, including any range between.

In some embodiments, the polyether is or comprises polyethylene glycol (PEG) or a derivative thereof.

Where appropriate, the abbreviation (PEG) is used in combination with a numeric suffix which indicates the average molecular weight of the PEG. A form of PEG or a PEG species is a PEG or PEG derivative with a specified average molecular weight.

As used herein, “PEG or derivatives thereof” refers to any compound including at least one polyethylene glycol moiety. PEGs exist in linear forms and branched forms comprising multi-arm and/or grafted polyethylene glycols. A PEG derivative may further comprise a functional group. A PEG derivative may be mono-, di-, or multifunctional polyethylene glycol.

Exemplary functional groups include, but are not limited to, the following: a hydroxyl, a carboxyl, a thiol, an amine, a phosphate, a phosphonate, a sulfate, a sulfite, a sulfonate, a sulfoxide, a sulfone, an amide, an ester, a ketone, an aldehyde, a cyano, an alkyne, an azide, and an alkene, or a combination thereof.

In some embodiments, the linker has an average MW (weight average) between 800 to 5,000 Da, including any range between.

In some embodiments, the linker has an average MW between 800 and 2,000 Da, between 800 and 1,000 Da, between 800 and 1,500 Da, between 800 and 900 Da, between 900 and 1,000 Da, between 1,000 and 1,100 Da, between 1000 and 1,200 Da, between 800 and 1,200 Da, between 1,000 and 3,000 Da, between 1,000 and 5,000 Da, between 1,000 and 7,000 Da, between 1,000 and 10,000 Da, between 2,000 and 3,000 Da, between 2,000 and 5,000 Da, between 2,000 and 7,000 Da, between 2,000 and 10,000 Da, between 3,000 and 5,000 Da, between 3,000 and 7,000 Da, between 3,000 and 10,000 Da, between 5,000 and 7,000 Da, between 5,000 and 10,000 Da, between 7,000 and 10,000 Da including any range between. Each possibility represents a separate embodiment.

According to some embodiments, the linker has an average MW of at least 800 Da, at least 900 Da, at least 800 Da, at least 1,000 Da, at least 1,200 Da, at least 1,500 Da, at least 2,000 Da, including any range between. Each possibility represents a separate embodiment. According to some embodiments, the polymer (and/or the linker) has an MW of at most 2,000 Da, at most 3,000 Da, at most 4,000 Da, at most 5,000 Da, and at most 7,000 Da. Each possibility represents a separate embodiment.

In some embodiments, the linker of the invention further comprises a bond or a functional group (e.g., an amide bond, an ester bond, a thioester bond, a disulfide bond, alkyl, a urea bond, a phosphate group, including any derivative or a combination thereof). In some embodiments, the first end and/or the second end of the linker each independently comprise one or more bonds.

In some embodiments, the first end of the linker is covalently bound to the fatty acid via a first bond; and the second end is covalently bound to the PSMA ligand via a second bond, wherein each first bond and the second bond is as described herein. In some embodiments, the first bond and the second bond is or comprises a click reaction product (e.g., a covalent linkage such as a cyclization reaction product and/or a succinimide-thioether moiety formed via a click reaction).

Click reactions are well-known in the art and comprise inter alia Michael addition of maleimide and thiol (resulting in the formation of a succinimide-thioether); azide-alkyne cycloaddition; Diels-Alder reaction (e.g., direct and/or inverse electron demand Diels Alder); dibenzyl cyclooctyne 1,3-nitrone (or azide) cycloaddition; alkene tetrazole photo click reaction, etc.

In some embodiments, the first bond and the second bond is or comprises an amide bond, an ester bond, a thioester bond, a disulfide bond, phosphate, etc.

In some embodiments, the conjugate of the invention is represented by Formula 2:

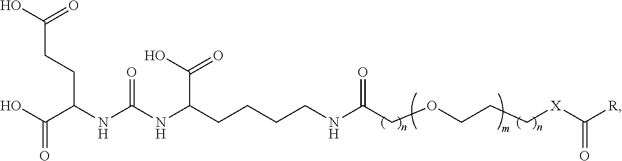

including any salt, and any stereoisomer thereof; wherein R is as described herein; X represents a heteroatom (e.g. O, N, NH, S), a bond, or a click reaction product; each n is independently 0, or between 1 and 10; and m is between 2 and 100, including any range between.

In some embodiments, X is NH. In some embodiments, each n is independently 0, or 1. In some embodiments, m is between 30 and 100, including any range between.

In some embodiments, the conjugate of the invention is capable of spontaneously undergoing micellization in an aqueous solution. In some embodiments, above a critical micellar concentration in an aqueous solution, the conjugate spontaneously undergoes micellization.

In some embodiments, the conjugate of the invention comprises a tracer covalently or coordinatively bound thereto. In some embodiments, the tracer is a radiolabel. In some embodiments, the tracer is a radioactive isotope. In some embodiments, the tracer is a PET-tracer (i.e. a positron emitting isotope, such as C-11, F-18, Ga-68, Lu-177, Cu-64, etc.). In some embodiments, the tracer is bound to the linker. In some embodiments, the tracer is bound to the spacer. In some embodiments, the tracer is a metal cation (e.g. Ga-68, Lu-177, Cu-64), wherein the tracer is coordinatively bound to the chelator. In some embodiments, the tracer is C-11 or F-18, wherein the tracer is covalently bound to the linker or to the spacer. Various methods for radioactively labeling the conjugates are well-known in the art.

In some embodiments, the tracer is a contrast agent (e.g. MRI-, CT-, SPECT-contrast agent). In some embodiments, the tracer is a fluorophore. In some embodiments, the tracer is a multimodal moiety comprising a radioactive isotope and at least one contrast agent.

### Particles

In another embodiment, the present invention further provides a nanoparticle encapsulating an inner liquid core (also referred to herein as “a core”). In some embodiments, the nanoparticle comprises a hydrophobic core and an amphiphilic shell, wherein the hydrophobic core is in a liquid (or fluid) state at a temperature between 10 and 100° C., between 10 and 50° C., between 20 and 100° C., between 20 and 50° C., including any range between. In some embodiments, the core comprises a water immiscible compound. In some embodiments, the core comprises a liquid oil. In some embodiments, the liquid oil comprises a plant oil. In some embodiments, the nanoparticle of the invention comprises the conjugate disclosed herein. In some embodiments, the liquid core is encapsulated by the shell comprising the conjugates of the invention. In some embodiments, the shell further comprises a PEG-ylated fatty acid.

In some embodiments, the PEG-ylated fatty acid comprises C5-C30 fatty acid covalently bound to PEG. In some embodiments, the C5-C30 fatty acid is bound to the PEG via a spacer. In some embodiments, PEG is as described herein.

In some embodiments, the PEG-ylated fatty acid is represented by Formula:

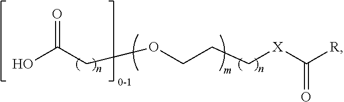

wherein m, n, X and R are as described hereinabove.

In some embodiments, the PEG-ylated fatty acid is represented by Formula:

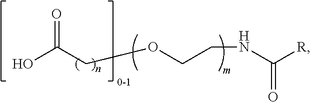

### or by Formula

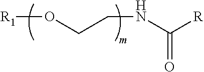

wherein m, n, X and R are as described hereinabove, and wherein R1 is H or an C1-C10 alkyl.

In some embodiments, a weight ratio between the PEG-ylated fatty acid and the conjugate of the invention within the nanoparticle or within the composition disclosed herein is between 100.000:1 and 10:1, between 10.000:1 and 10:1, between 1.000:1 and 10:1, between 5.000:1 and 10:1, between 50.000:1 and 10:1, between 500:1 and 10:1, between 100:1 and 10:1, between 100:1 and 1:1, including any range between.

The term “core”, as used herein, refers to the central portion of the nanoparticle, with a different composition than the shell. In some embodiments, the core is enclosed by the shell. In some embodiments, the core is bound to the inner portion of the shell. In some embodiments, the shell form a layer around the core.

In some embodiments, the plant oil is a natural triacylglyceride. In some embodiments, the plant oil is selected from medium-chain triacylglyceride (MCT) oil, and short-chain triacylglyceride (SCT) oil. In some embodiments, the plant oil is a terpenoid oil.

Non-limiting examples of plant oils include but are not limited to: an olive oil, a sunflower oil, a safflower oil, a corn oil, a canola oil, a wheat germ oil, a peanut oil, a soy oil, a coconut oil, a vegetable oil, an orange oil, a citrus oil, limonene, or any combination thereof. In some embodiments, the plant oil is a refined olive oil.

In some embodiments, the liquid oil comprises a fatty acid. In some embodiments, the fatty acid comprises a C7-C30, C7-C20, C10-C30, C15-C30, C15-C20 fatty acid, including any range between. In some embodiments, the liquid oil comprises an unsaturated fatty acid comprising one or more unsaturated bond(s). In some embodiments, the liquid oil is or comprises an oleic acid.

In some embodiments, the particle is a solid. In some embodiments, the nanoparticle and/or the core comprises a hydrophobic compound.

In some embodiments, the hydrophobic compound is dissolved or dispersed in the liquid oil. In some embodiments, the hydrophobic core comprises the liquid oil and the hydrophobic compound. In some embodiments, the hydrophobic compound is homogenously distributed within the core. In some embodiments, at least 80%, at least 90%, at least 95%, at least 99% by weight of the hydrophobic compound is stably encapsulated within the nanoparticle. The term “stably” refers to the physical stability of the nanoparticle, being substantially devoid of: disintegration, release of the active agent, changes in one or more physical properties, when stored under appropriate conditions for a time period between 1 day and 2 years, 1 d and 1 year, including any range between. In some embodiments, the term “stable” refers to physical and chemical stability of the carrier (such as being substantially devoid of phase separation, agglomeration, disintegration, and/or substantially retaining the initial loading of the active agent) under appropriate storage conditions. In some embodiments, the term “stable” refers to physical and chemical stability of the carrier within an aqueous solution (e.g., dispersion stability). In some embodiments, the term appropriate storage conditions comprise an ambient atmosphere and a temperature between 5 and 45°, between 5 and 15°, between 15 and 45°, and between 20 and 45°, including any range between.

In some embodiments, the shell is a single layer shell. In some embodiments, the shell is a double layer shell. In some embodiments, the shell comprises a first layer and a second layer.

In some embodiments, the single layer shell comprises an inner portion facing the core and an outer portion. In some embodiments, the outer portion faces an ambient (e.g. atmospheric air, or an aqueous solution). In some embodiments, the conjugate of the invention stabilizes the liquid core.

In some embodiments, the hydrophobic compound is a pharmaceutically active agent. In some embodiments, the pharmaceutically active agent (also used herein as the “active agent”) is an oil soluble compound. In some embodiments, the active agent is characterized by an oil/fat solubility of at least 1 g/L, at least 5 g/L, at least 10 g/L, at least 30 g/L, at least 50 g/L, at least 100 g/L, between 1 and 100 g/L, between 1 and 50 g/L, including any range between.

In another embodiment, the present invention further provides that in its free form, the bioactive compound has maximal aqueous solubility below 5 g/l (water). In another embodiment, the present invention further provides that in its free form, the bioactive compound has maximal aqueous solubility below 3 g/l (water). In another embodiment, the present invention further provides that in its free form, the bioactive compound has maximal aqueous solubility below 2 g/l (water). In another embodiment, the present invention further provides that in its free form, the bioactive compound has maximal aqueous solubility below 1.5 g/l (water). In another embodiment, the present invention further provides that in its free form, the bioactive compound has maximal aqueous solubility below 0.5 g/l (water). In another embodiment, the present invention further provides that in its free form, the bioactive compound has maximal aqueous solubility below 0.1 g/l (water).

In another embodiment, the bioactive compound is or comprises an anti-cancer agent.

The phrase “anticancer agent” or “anticancer drug”, as used herein, describes a therapeutically active agent that directly or indirectly kills cancer cells or directly or indirectly inhibits, stops, halts, or reduces the proliferation of cancer cells. Anti-cancer agents include those that result in cell death and those that inhibit cell growth, proliferation and/or differentiation. In some embodiments, the anti-cancer agent is selectively toxic against certain types of cancer cells but does not affect or is less effective against normal cells. In some embodiments, the anti-cancer agent is a cytotoxic agent.

Examples of cancer therapeutic agents include, e.g., but are not limited to Abiraterone, Acitretin, Aldesleukin, Alemtuzumab, Amifostine, Amsacrine, Anagrelide, Anastrozole, Arsenic, Asparaginase, Asparaginase Erwinia, Axitinib, azaCITItidine, BCG, Bendamustine, Bevacizumab, Bexarotene, Bicalutamide, Bleomycin, Bortezomib, Brentuximab, Bromocriptine, Buserelin, Busulfan, Cabazitaxel, Cabergoline, Capecitabine, CARBOplatin, Carmustine, Cetuximab, Chlorambucil, ClSplatin, Cladribine, Clodronate, Crizotinib, Cyclophosphamide, CycloSPORINE, Cytarabine, Dacarbazine, Dactinomycin, Dasatinib, DAUNOrubicin, Degarelix, Denosumab, Dexamethasone, Dexrazoxane, DOCEtaxel, DOXOrubicin, DOXOrubicin pegylated liposomal, Enzalutamide, Epirubicin, Eribulin, Erlotinib, Estramustine, Etoposide, Everolimus, Exemestane, Filgrastim, Fludarabine, Fluorouracil, Flutamide, Fulvestrant, Gefitinib, Gemcitabine, Goserelin, Hydroxyurea, IDArubicin, Ifosfamide, Imatinib, Iniparib, Interferon alfa-2b, Ipilimumab, Irinotecan, Ixabepilone, Lambrolizumab, Lanreotide, Lapatinib, Lenalidomide, Letrozole, Leucovorin, Leuprolide, Lomustine, Mechlorethamine, medroxyPROGESTERone, Megestrol, Melphalan, Mercaptopurine, Mesna, Methotrexate, mitoMYCIN, Mitotane, mitoXANTRONE, Nilotinib, Nilutamide, Octreotide, Ofatumumab, Oxaliplatin, PACLitaxel, ACLitaxel nanoparticle, albumin-bound (nab), Pamidronate, Panitumumab, Pazopanib Pemetrexed, Pertuzumab, Porfimer, Procarbazine, Quinagolide, Raltitrexed, Reovirus Serotype 3—Dearing Strain, riTUXimab, Romidepsin, Ruxolitinib, SORAfenib, Streptozocin, SUNItinib, Tamoxifen, Temozolomide, Temsirolimus, Teniposide, Testosterone, Thalidomide, Thioguanine, Thiotepa, Thyrotropin alfa, Tocilizumab, Topotecan, Trastuzumab (HERCEPTIN®), Trastuzumab, Emtansine (KADCYLA®), Treosulfan, Tretinoin, Vemurafenib, vinBLAstine, vinCRIstine and Vinorelbine.

Examples of chemotherapeutic agents used as a therapeutic agent include, e.g., but are not limited to, e.g., alkylating agents (e.g., cyclophosphamide, ifosfamide, melphalan, chlorambucil, aziridines, epoxides, alkyl sulfonates), cisplatin and its analogues (e.g., carboplatin, oxaliplatin), antimetabolitites (e.g., methotrexate, 5-fluorouracil, capecitabine, cytarabine, gemcitabine, fludarabine), topoisomerase interactive agents (e.g., camptothecin, irinotecan, topotecan, etoposide, teniposide, doxorubicin, daunorubicin), antimicrotubule agents (e.g., vinca alkaloids, such as vincristine, vinblastine, and vinorelbine; taxanes, such as paclitaxel and docetaxel), interferons, interleukin-2, histone deacetylase inhibitors, monoclonal antibodies, estrogen modulators (e.g., tamoxifen, toremifene, raloxifene), megestrol, aromatase inhibitors (e.g., letrozole, anastrozole, exemestane, octreotide), octreotide, anti-androgens (e.g., flutamide, casodex), kinase and tyrosine inhibitors (e.g., imatinib (STI571 or Gleevac); gefitinib (Iressa); and erlotinib (Tarceva), etc. See, e.g. Cancer: Principles and Practice of Oncology, 7th Edition, Devita et al, Lippincott Williams & Wilkins, 2005, Chapters 15, 16, 17, and 63).

In another embodiment, the present invention further provides a composition comprising a plurality of nanoparticles of the invention.

In some embodiments, a size (or a mean hydrodynamic diameter) of the nanoparticles is between 10 and 1000 nm, between 30 and 300 nm, between 10 and 20 nm, between 20 and 30 nm, between 30 and 50 nm, between 50 and 60 nm, between 60 and 70 nm, between 70 and 100 nm, between 100 and 200 nm, between 100 and 150 nm, between 150 and 200 nm, between 50 and 200 nm, between 200 and 300 nm, between 300 and 500 nm, between 500 and 1000 nm, including any range or value therebetween. In some embodiments, a size (or a mean hydrodynamic diameter) of the nanoparticles encapsulating the active agent is between about 50 and about 200 nm, between about 80 and about 200 nm, between about 100 and about 200 nm, including any range or value therebetween.

In some embodiments, nanoparticles of the invention are further characterized by a polydispersity index between about 1.0 and 1.5, between about 1.0 and 1.1, between about 1.0 and 1.01, between about 1.0 and 1.2, between about 1.0 and 1.3, between about 1.0 and 1.5, including any range or value therebetween.

In another embodiment, the nanoparticles of the invention are characterized by a negative zeta potential (e.g., measure at a pH between about 6 and 8). In some embodiments, the nanoparticles of the invention are characterized by a zeta potential ranging between −0.1 and −50 mV, between −0.5 and −50 mV, between −0.5 and −40 mV, between −10 and −50 mV, between −1 and −50 mV, between −20 and −50 mV, including any range between.

In some embodiments, a weight ratio between the liquid oil and the PEG-ylated fatty acid within the nanoparticle or the composition is between about 1:1 and about 1:5, between about 1:1 and about 1:3, between about 1:1 and about 1:2, between about 1:1 and about 1:1.5, including any range between.

In some embodiments, a molar ratio between the active agent and the PEG-ylated fatty acid within the nanoparticle or within the composition is between about 0.1:1 and about 2:1, between about 0.2:1 and about 2:1, between about 0.2:1 and about 1:1, between about 0.1:1 and about 0.8:1, between about 0.2:1 and about 0.8:1, including any range between.

In some embodiments, the composition is a solid comprising a plurality of solid nanoparticles. In some embodiments, the composition is a liquid composition comprising a plurality of nanoparticles of the invention. In some embodiments, the liquid composition is an aqueous solution comprising a plurality of nanoparticles dispersed therewithin. In some embodiments, the nanoparticles are stably dispersed within the liquid composition (e.g., being substantially devoid of: aggregates and/or disintegration of the nanoparticles). In some embodiments, the nanoparticles within the liquid composition are stably encapsulating the bioactive compound (e.g., being substantially devoid of disintegration of the nanoparticles and leakage of the bioactive compound, and/or the bioactive compound substantially maintains its biological activity, as compared to a non-encapsulated bioactive compound). In some embodiments, the liquid composition is stable for at least 24 h, at least 48 h, at least three days (d), at least 7 d, at least 30 d, at least 60 d, at least 150 d, and at least one year (y) including any range between, when stored under normal (or appropriate) storage conditions.

In some embodiments, concertation of the nanoparticles within the liquid composition is between 10 nm and 10 μM, between 10 nm and 10 mM, between 10 nm and 1 mM, including any range between.

In another embodiment, the present invention further provides a composition comprising the nanoparticles in an aqueous solution. In another embodiment, the present invention further provides that the composition comprising the nanoparticles is a nanoemulsion. In another embodiment, the present invention further provides that the aqueous solution is a transparent aqueous liquid.

In another embodiment, the aqueous solution is transparent. In another embodiment, the aqueous solution comprises at least 70% by weight water. In another embodiment, the aqueous solution comprises at least 75% by weight water. In another embodiment, the aqueous solution comprises at least 85% by weight water. In another embodiment, the aqueous solution comprises at least 90% by weight water. In another embodiment, the aqueous solution comprises at least 95% by weight water. In another embodiment, the aqueous solution comprises at least 98% by weight water.

In another embodiment, the present invention further provides that the bioactive compound is present at a concentration of 0.01 microgram/ml to 10 mg/ml within the composition of the invention. In another embodiment, the present invention further provides that the bioactive compound is present at a concentration of 0.5 mg/ml to 5 mg/ml. In another embodiment, the present invention further provides that the bioactive compound is present at a concentration of 1 microgram/ml to 1 mg/ml. In another embodiment, the present invention further provides that the bioactive compound is present at a concentration of 100 microgram/ml to 1 mg/ml. In another embodiment, the present invention further provides that the bioactive compound is present at a concentration of 100 microgram/ml to 0.5 mg/ml. In another embodiment, the present invention further provides that the bioactive compound is present at a concentration of 0.5 mg/ml to 1 mg/ml. In another embodiment, the present invention further provides that the bioactive compound is present at a concentration of 0.1 mg/ml to 1 mg/ml. In another embodiment, the present invention further provides that the bioactive compound is present at a concentration of 0.5 mg/ml to 1 mg/ml.

In another embodiment, the present invention further provides that the nanoparticles and/or nanocapsules or any composition comprising the nanoparticles and/or nanocapsules is/are devoid of a surfactant. In another embodiment, the present invention further provides that the nanoparticles and/or nanocapsules or any composition comprising the nanoparticles and/or nanocapsules is/are devoid of a low molecular weight surfactant. In another embodiment, the nanoparticles of the invention are composed essentially of the conjugate of the invention, the liquid oil, and the active agent, as disclosed herein.

In another embodiment, a composition of the invention is devoid of an organic solvent. In another embodiment, a composition of the invention is devoid of alcohol. In another embodiment, a composition of the invention in the form of a solution is free of a low molecular weight emulsifier. In another embodiment, a composition of the invention comprises a water miscible solvent such as ethanol or DMSO in a trace amount.

In another embodiment, a composition of the invention comprises less than 5% in weight a water miscible solvent such as ethanol.

In some embodiments, the liquid composition is a pharmaceutical composition, comprising a pharmaceutically effective amount of the nanoparticles of the invention and/or a pharmaceutically effective amount of the bioactive compound. In some embodiments, the pharmaceutical composition comprises a plurality of the nanoparticles of the invention and a pharmaceutically acceptable carrier.

As used herein, a “pharmaceutically acceptable formulation,” “pharmaceutical composition,” or “pharmaceutically acceptable composition” may include any of a number of carriers such as solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Pharmaceutical compositions containing the presently described nanoparticles as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

A composition may comprise different types of carriers depending on whether it is to be administered in solid, liquid, or aerosol form and whether it needs to be sterile for such routes of administration as injection. A person of ordinary skill in the art would be familiar with techniques for generating sterile solutions for injection or application by any other route. Sterile injectable solutions are prepared by incorporating the active compounds in the required amount in an appropriate solvent with various other ingredients familiar to a person of skill in the art.

The carrier may comprise, in total, from about 0.1% to about 99.99999% by weight of the pharmaceutical compositions presented herein.

According to some embodiments, the pharmaceutical composition is formulated for systemic administration. According to some embodiments, the pharmaceutical composition is formulated for topical administration.

The compositions contemplated herein may take the form of solutions, suspensions, emulsions, combinations thereof, or any other pharmaceutical acceptable composition as would commonly be known in the art.

In some embodiments, the carrier is a solvent. For a non-limiting example, the composition may be disposed of in the solvent. Such a solvent includes any suitable solvent known in the art, such as water, saline, or phosphate-buffered saline.

The formulation of the composition may vary depending upon the route of administration. For parenteral administration in an aqueous solution, for example, the solution should be suitably buffered if necessary, and the liquid diluent first rendered isotonic with sufficient saline or glucose. Sterile aqueous media which can be employed will be known to those of skill in the art in light of the present disclosure.

Supplementary active ingredients can also be incorporated into the compositions. For human administration, preparations should meet sterility and general safety and purity standards as required by FDA Office of Biologics standards. Administration may be by any known route.

In certain embodiments, a pharmaceutical composition includes at least about 0.01 g to about 5 g of the particle disclosed herein per kilogram of a subject.

The pharmaceutical composition may comprise various antioxidants to retard the oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof. The composition must be stable under the conditions of manufacture and storage and preserved against the contaminating action of microorganisms, such as bacteria and fungi.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. In many cases, it will be preferable to include isotonic agents, such as, for example, sugars, sodium chloride, or combinations thereof.

In other embodiments, nasal solutions or sprays, aerosols, or inhalants may be used. Nasal solutions are usually aqueous solutions designed to be administered to the nasal passages in drops or sprays.

Solid compositions for oral administration are also contemplated. In these embodiments, the solid composition may comprise, for example, solutions, suspensions, emulsions, tablets, pills, capsules, sustained release formulations, buccal compositions, troches, elixirs, suspensions, syrups, or combinations thereof.

Sterile injectable solutions are prepared by incorporating the active compounds (e.g., nanoparticles) in the required amount in the appropriate solvent with various other ingredients enumerated above. The liquid medium should be suitably buffered if necessary, and the liquid diluent first rendered isotonic prior to injection with sufficient saline or glucose.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art.

### Therapeutic and Diagnostic Use of the Composition

According to some embodiments, there is provided a method for introducing an agent into a PSMA-expressing cell.

According to some embodiments, there is provided a method for treating a PSMA-related disease or disorder in a subject in need thereof.

As used herein, the term “PSMA-related disease or disorder”, refers to any disease, condition, disorder, pathology, or any combination thereof, wherein a PSMA gene or a protein encoded therefrom is involved, induces, initiates, propagates, determines, or any combination or equivalent thereof, in the pathogenesis, pathophysiology, or both.

In some embodiments, PSMA refers to: glutamate carboxypeptidase II (GCP-II), N-acetyl-α-linked acidic dipeptidase I, or folate hydrolase.

Nucleic acid sequence and/or amino acid sequence of PSMA, would be apparent to one of ordinary skill in the art.

Non-limiting examples of such nucleic acid sequence pare provided in RefSec (mRNA) numbers: NM_001014986, NM_001193471, NM_001193472, NM_001193473, and NM_004476.

Non-limiting examples of such nucleic acid sequence pare provided in RefSec (protein) numbers: NP_001014986, NP_001180400, NP_001180401, NP_001180402, and NP_004467.

In some embodiments, the method comprises contacting the cell with an effective amount of the conjugate disclosed herein, and an agent.

In some embodiments, the agent is encapsulated by or within the conjugate.

In some embodiments, the method comprises contacting the cell with an effective amount of the conjugate disclosed and an agent being encapsulated by or within the conjugate.

In some embodiments, the method comprises contacting the cell with an effective amount of contacting the cell with an effective amount of a composition comprising a plurality of nanoparticles, wherein each of the plurality of nanoparticles comprises a core and a shell, wherein: the shell comprises a conjugate as disclosed herein; and a core comprises a liquid oil.

In some embodiments, the liquid oil comprises or further comprises the agent, such as a pharmaceutically active agent.

In some embodiments, the agent is a dye agent. In some embodiments, the agent is a detectable agent.

In some embodiments, the agent is a fluorescent agent or comprising a fluorophore.

In some embodiments, agent is a radioactive agent or comprises a radioactive isotope.

In some embodiments, the method further comprises a step comprising detecting or determining the presence, the amount, or both, of the agent in the subject or in a sample obtained or derived therefrom.

In some embodiments, the method comprises administering to the subject a therapeutically effective amount of a pharmaceutical composition as disclosed herein.

In some embodiments, the cell comprises or is a prostate gland cell, or a progenitor cell thereof.

In some embodiments, the cell is a prostate cancer or cancerous cell.

In some embodiments, the agent is a oil-soluble compound. In some embodiments, the agent is a hydrophobic agent. In some embodiments, the agent is insoluble in water per se. In some embodiments, solubility or insolubility is under at least one physiologic condition selected from: pH, temperature, osmolarity, or any combination thereof. In some embodiments, physiologic conditions refer to or correspond to conditions suitable for mammalian cells or organisms.

In some embodiments, the cell is obtained or derived from a subject afflicted with cancer. In some embodiments, the cell is obtained or derived from a subject afflicted with prostate cancer or prostate gland cancer (both terms are used herein interchangeably).

In some embodiments, PSMA-related disease or disorder is a cell-proliferation related disease.

As used herein, the term “cell proliferation related disease” refers to any disease or condition associated therewith comprising or characterized by increased, enhanced, unregulated, dysregulated, abnormal, excessive, or any combination thereof, of cell proliferation.

In some embodiments, a cell proliferation related disease comprises cancer.

In some embodiments, cancer comprises prostate cancer.

As used herein “cancer” or “pre-malignancy” are diseases associated with cell proliferation. Non-limiting types of cancer include carcinoma, sarcoma, lymphoma, leukemia, blastoma and germ cells tumors. In one embodiment, carcinoma refers to tumors derived from epithelial cells. In one embodiment, sarcoma refers of tumors derived from mesenchymal cells. In one embodiment, lymphoma refers to tumors derived from hematopoietic cells that leave the bone marrow and tend to mature in the lymph nodes. In one embodiment, leukemia refers to tumors derived from hematopoietic cells that leave the bone marrow and tend to mature in the blood. In one embodiment, blastoma refers to tumors derived from immature precursor cells or embryonic tissue. In one embodiment, germ cell tumors refers to tumors derived from germ cells. In one embodiment, nongerminomatous or nonseminomatous tumors refers to pure and mixed germ cells tumors.

In some embodiments, at least one cell of the prostate cancer is characterized by increased expression, abundance, or both, of PSMA transcript, protein product thereof, or both, compared to a control.

In some embodiments, one or more cells of the prostate cancer is characterized by increased expression, abundance, or both, of PSMA transcript, protein product thereof, or both, compared to a control.

In some embodiments, a plurality of cells of the prostate cancer are characterized by increased expression, abundance, or both, of PSMA transcript, protein product thereof, or both, compared to a control.

In some embodiments, a control comprises a non-treated subject or a sample derived therefrom. In some embodiments, a control comprises subject being afflicted with cancer or prostate cancer that is not being treated according to the method disclosed herein or a sample derived therefrom. In some embodiments, a control comprises the same subject before being treated according to the method disclosed herein or a sample derived therefrom.

In some embodiments, a plurality comprises any integer equal to or greater than 2.

In some embodiments, the method further comprises a step preceding the administering, comprising determining expression, abundance, or both, of PSMA transcript, protein product thereof, or both, in a sample derived or obtained from the subject.

Methods for determining PSMA expression are common and would be apparent to one of ordinary skill in the art. Non-limiting examples for methods of determining expression include, but are not limited to, RT-PCR, real time RT-PCR, next generation sequencing, western blot, dot blot, enzyme linked immunosorbent assay (ELISA), and other.

In some embodiments, an expression, abundance, or both, of the PSMA transcript, protein product thereof, or both, being above a predetermined threshold, is indicative of the subject being suitable for the administering.

In some embodiments, an expression, abundance, or both, of the PSMA transcript, protein product thereof, or both, being below a predetermined threshold, is indicative of the subject being unsuitable for the administering.

In some embodiments, the sample comprises at least one cell of the prostate cancer.

In some embodiments, the sample comprises one or more cells of the prostate cancer.

some embodiments, the sample comprises a plurality of cells of the prostate cancer.

In some embodiments, the terms cancer and tumor, are used herein interchangeably.

In some embodiments, treating comprises: reducing the number of proliferating cells of the cancer, reducing the rate of cell proliferation of cells of the cancer, reducing the survival or viability of cells of the cancer, or any combination thereof.

According to some embodiments, the pharmaceutical composition is for use in the prevention or treatment of a disease in a subject in need thereof. According to some embodiments, the pharmaceutical composition is for use in the treatment of a disease in a subject in need thereof In some embodiments, there is provided a method for treating or reducing at least one symptom associated with the disease in the subject, the method comprising administering a therapeutically effective amount of the pharmaceutical composition of the invention to the subject. In some embodiments, the method is for a targeted delivery of the active agent to the target site within the body of the subject, as disclosed herein. In some embodiments, the targeted delivery is so as to induce an enhanced accumulation of the active agent within the target site, wherein enhanced is as described herein.

In another embodiment, a subject is a human. In another embodiment, a subject is an infant. In another embodiment, a subject is a toddler. In another embodiment, a subject is a pet. In another embodiment, a subject is a farm animal. In another embodiment, a subject is a rodent. In some embodiments, the subject is a human subject. In some embodiments, the subject is at risk of being afflicted with a disease, a disorder, or a medical condition. In some embodiments, the subject is diagnosed with a disease, a disorder, or a medical condition. In some embodiments, the subject is diagnosed with a genetic disorder. As used herein, a subject at risk of being afflicted with a disease, a disorder, or a medical condition, is a subject that presents one or more signs or symptoms indicative of a disease, a disorder, or a medical condition or is being screened for a disease, a disorder, or a medical condition (e.g., during a routine physical). A subject at risk of being afflicted with a disease, a disorder, or a medical condition, may also have one or more risk factors. A subject at risk of being afflicted with a disease, a disorder, or a medical condition encompasses an individual that has not been previously tested for the disease, disorder, or medical condition. However, a subject at risk of being afflicted with a disease, a disorder, or a medical condition, also encompasses an individual who has received a preliminary diagnosis but for whom a confirmatory test (e.g., biopsy and/or histology) has not been done or for whom the stage of the disease, disorder, or medical condition is not known. The term further includes people who once had the disease, disorder, or medical condition (e.g., an individual in remission).

A subject at risk of being afflicted with a disease, disorder, or medical condition may be diagnosed as having or alternatively found not to have the disease, disorder, or medical condition.

As used herein, a subject diagnosed with a disease, disorder, or medical condition, may be diagnosed using any suitable method, including but not limited to biopsy, x-ray, blood test, and the diagnostic methods of the present invention. A “preliminary diagnosis” is one based only on visual (e.g., CT scan or the presence of a lump) and antigen tests.

In some embodiments, the subject is afflicted with a disease, disorder, or medical condition, and the imaging method is used to determine the stage of the disease, disorder, or medical condition. In some embodiments, the subject afflicted with a disease, disorder, or medical condition, was treated with a drug, and the imaging method is used for follow-up of the treatment.

As used herein, the terms “treatment”, “treating”, or “ameliorating” of a disease, disorder, or condition, refer to the alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, the present invention provides for a method of administering a biologically active agent for the prevention or treatment of a disease in a subject in need thereof, the method comprising administering to the subject the pharmaceutical composition described herein.

In some embodiments, the present invention provides a theranostic method. The method comprises the steps of administering to a subject in need thereof the pharmaceutical composition of the invention and imaging a target site of the subject to determine whether the nanoparticles accumulated in the target site of the subject. In some embodiments, the target site is a site in the brain of the subject.

In some embodiments, administering the pharmaceutical composition to the subject can be done by using any method known to those of ordinary skill in the art. The mode of administering may vary based on the application. For example, the mode of administration may vary depending on the particular cell, tissue, organ, portion of the body, or subject to be imaged. For example, administering the composition may be done intravenously, intracerebrally, intracranially, intrathecally, intracerebroventricular, into the substantia nigra or the region of the substantia nigra, intradermally, intraarterially, intraperitoneally, intralesionally, intratracheally, intranasally, intramuscularly, intraperitoneally, subcutaneously, orally, topically, locally, by inhalation (e.g., aerosol inhalation), by injection, by infusion, by continuous infusion, by localized perfusion bathing target cells directly, via a catheter, via a lavage, or by other method or any combination of the forgoing as would be known to one of ordinary skill in the art.

In some embodiments, the pharmaceutical composition is administered intravenously.

Upon formulation, compositions will be administered in a manner compatible with the dosage formulation and in such amount as is effective. For example, the nanoparticles may be administered in such an amount that is effective for a particular imaging application desired.

An effective amount of the pharmaceutical composition is determined based on the intended goal, for example, based on the imaging method and the subject or portion of a subject to be imaged. The quantity to be administered may also vary based on the particular route of administration to be used. The composition is preferably administered in a “safe and effective amount.” As used herein, the term “safe and effective amount” refers to the quantity of a composition which is sufficient for the intended goal (e.g., imaging) without undue adverse side effects (such as toxicity, irritation, or allergic response).

In some embodiments, imaging of the target site is performed by an imaging technique that utilizes penetrating radiation. According to some embodiments, the imaging technique is selected from the group comprising magnetic resonance imaging (MM), computed tomography imaging (CT), X-ray imaging, positron emission tomography (PET), single-photon emission computed tomography (SPECT), and ultrasound (US).

In some embodiments, the imaging step is performed 0.5 to 96 hours post the administering step.

In some embodiments, the method comprises the step of determining whether the nanoparticles accumulated in the target site of the subject. In some embodiments, the treatment decision may be not to administer therapy. In some embodiments, the analysis of the imaging data is used for deciding on a route of treatment adequate to the patient. In some embodiments, deciding on a route of treatment adequate to the patient depends, for example, on the stage of the disease, disorder, or medical condition, as well as on the health state of the patient. In some embodiments, the route of treatment includes one or more protocols of treatment selected from the group comprising: intravenous, intranasal, intraperitoneal, intramuscular and subcutaneous, and any other biological or inorganic product intended for treatment. In some embodiments, treatment is administered subsequent to the imaging. In some embodiments, treatment is administered to the subject in real-time while imaging the subject.

In some embodiments, imaging and treating the subject are performed simultaneously. In some embodiments, the biologically active molecule may be activated in the subject target site subsequent to imaging.

According to some embodiments, there is provided a kit comprising a conjugate as disclosed herein, and an agent as disclosed herein.

In some embodiments, the kit is used in a method for preparing a composition as disclosed herein. In some embodiments, the composition is a pharmaceutical composition. In some embodiments, the composition is a diagnostic composition.

In another embodiment, the invention further provides a kit comprising the nanoparticles of the invention in liquid or dry form and dosing, mixing, and/or formulating instructions. In another embodiment, the invention further provides a kit comprising the nanoparticles of the invention and dosing, mixing, and/or formulating instructions with an aqueous solution as described herein. In another embodiment, the invention further provides a kit comprising the nanoparticles, an aqueous solution as described herein and dosing, mixing, and/or formulating instructions.

In one embodiment, compositions of the present invention are presented in a pack or dispenser device, such as an FDA approved kit, which contain one or more unit dosage forms containing the nanoparticles. In one embodiment, the pack, for example, comprise metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals and/or nutraceuticals, which notice is reflective of approval by the agency of the form of the compositions or human or veterinary administration. Such notice, in one embodiment, is labeling approved by the U.S. Food and Drug Administration for prescription drugs or of an approved product insert.

In one embodiment, toxicity and therapeutic efficacy of the nanoparticles or described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

In one embodiment, depending on the severity and responsiveness of the condition to be treated, dosing can be of a single or a plurality of administrations, with course of treatment lasting from several days to several weeks or until cure is affected, or diminution of the disease state is achieved.

In one embodiment, the amount of a composition to be administered will, of course, be dependent on the subject being treated, the severity of the affliction, the manner of administration, the judgment of the prescribing physician, etc.

In one embodiment, the nanoparticles of the present invention can be provided to the individual per se (as a powder for example). In one embodiment, the nanoparticles of the present invention can be provided to the individual as part of a pharmaceutical composition where it is mixed with a pharmaceutically acceptable carrier.

In one embodiment, a “pharmaceutical composition” refers to a preparation of one or more nanoparticles described herein with other chemical components such as physiologically suitable carriers and excipients. The purpose of a pharmaceutical composition is to facilitate administration of nanoparticles to an organism.

In one embodiment, the phrases “physiologically acceptable carrier” and “pharmaceutically acceptable carrier”, which are interchangeably used, refer to a carrier or a diluent that does not cause significant irritation to an organism and does not abrogate the biological activity and properties of the administered compound. An adjuvant is included under these phrases. In one embodiment, one of the ingredients included in the pharmaceutically acceptable carrier can be for example polyethylene glycol (PEG), a biocompatible polymer with a wide range of solubility in both organic and aqueous media (Mutter et al. (1979)).

In one embodiment, “excipient” refers to an inert substance added to a pharmaceutical composition to further facilitate administration of nanoparticles. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, PA, latest edition, which is incorporated herein by reference.

In one embodiment, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

Oral administration, in one embodiment, comprises a unit dosage form comprising solutions, suspensions, emulsions and the like. Such unit dosage forms comprise a safe and effective amount of the desired nanocapsules and/or nanoparticles.

Peroral compositions, in some embodiments, comprise liquid solutions, emulsions, suspensions, and the like.

In some embodiments, compositions for use in the methods of this invention comprise solutions or emulsions, which in some embodiments are aqueous solutions or emulsions comprising a safe and effective amount of the nanocapsules and/or nanoparticles of the present invention and optionally, other compounds. In some embodiments, the compositions comprise from about 0.01% to about 30% w/v of the nanoparticles of the invention, more preferably from about 0.1% to about 10%.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, the nanoparticles of the present invention are combined with an additional appropriate therapeutic agent or agents, prepared and applied as solutions, suspensions, or emulsions in a physiologically acceptable diluent with or without a pharmaceutical carrier.

In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

In one embodiment, injectables, of the invention are formulated in aqueous solutions. In one embodiment, injectables, of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in aqueous vehicles, and contain formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In another embodiment, the pharmaceutical composition is delivered in a controlled release system formulated for intravenous infusion, implantable osmotic pump, transdermal patch, or other modes of administration. In one embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). Other controlled release systems are discussed in the review by Langer (Science 249:1527-1533 (1990).

In some embodiments, the nanocapsules and/or nanoparticles are in powder form and possibly in kits for constitution with a suitable vehicle, e.g., sterile, pyrogen-free water-based solution or a beverage, before use. Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

### General

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure, or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, and “having” are interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

As used herein, the term “about” when combined with a value refers to plus and minus 10% of the reference value. For example, a length of about 1000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In some embodiments, the term “substituted” comprises more or more (e.g. 2, 3, 4, 5, 6, or more) substituents, wherein the substituent(s) is as described herein. The term “substituent”, as used herein comprises one or more substituents (e.g. 1, 2, 3, 4, 5, or 6), each independently selected from the group consisting of: C₁-C₆ alkyl, halo, —NO₂, —CN, —OH, —NH₂, carbonyl, —CONH₂, —CONR′₂, —CNNR₂, —CSNR₂, —CONH—OH, —CONH—NH₂, —NHCOR′, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R′, —OCOR′, —OCOR′, —OC(═O)OR′, —OC(═O)NR′, —OC(═S)OR′, —OC(═S)NR′, a heteroatom, an optionally substituted cycloalkyl, an optionally substituted heterocyclyl, or a combination thereof, wherein each R′ is independently selected from hydrogen, alkyl, cycloalkyl, alkenyl, aryl, heteroaryl (e.g. optionally bonded through a ring carbon, or through a heteroatom) or heterocyclyl (e.g. optionally bonded through a ring carbon, or through a heteroatom).

As used herein, the term “alkyl” describes an aliphatic hydrocarbon including straight chain and branched chain groups. In some embodiments, the alkyl group has 1 to 20 carbon atoms, between 1 and 10, between 1 and 5, between 5 and 10, between 10 and 15, between 15 and 20, including any range between.

In some embodiments, the alkyl encompasses a short alkyl and/or a long alkyl. In some embodiments, the alkyl has from 21 to 100 carbon atoms, or more. In the context of the present invention, a “long alkyl” is an alkyl having at least 20 carbon atoms in its main chain (the longest path of continuous covalently attached atoms). A short alkyl therefore has 20 or less (e.g. 2, 3, 4, 5, 6, 8, 10, 15, or 20) main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (e.g. optionally bonded through a ring carbon, or through a heteroatom) or heterocyclyl (e.g. optionally bonded through a ring carbon, or through a heteroatom) as defined herein.

In some embodiments, R′ and R″ are the same or different, wherein each of R′ and R″ is independently selected from hydrogen, alkyl, cycloalkyl, alkenyl, aryl, heteroaryl (e.g. optionally bonded through a ring carbon, or through a heteroatom) or heterocyclyl (e.g. optionally bonded through a ring carbon, or through a heteroatom) as defined herein.

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)-linking group, as these phrases are defined hereinabove, where R′ and R″ are as defined herein.

A “cyano” or “nitrile” group refers to a —CN group. The term “azo” or “diazo” describes an —N═NR′ end group or an —N═N-linking group, as these phrases are defined hereinabove, with R′ as defined hereinabove. The term “guanidine” describes a —R′NC(N)NR″R′″ end group or a —R′NC(N)NR″-linking group, as these phrases are defined hereinabove, where R′, R″ and R′″ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C3-8 heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3 -b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pryidyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C1-6-alkyl, C1-6-haloalkyl, C1-6-hydroxyalkyl, C1-6-aminoalkyl, C1-6-alkylamino, alkylsulfenyl, alkylsulfinyl, alkyl sulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C1-6-alkyl, C1-6-alkyl, hydroxy-C1-6-alkyl and amino-C1-6-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization - A Laboratory Course Manual” CSHL Press (1996); all of which are incorporated by reference. Other general references are provided throughout this document.

### Materials and Methods

### Materials

All chemicals were obtained from Sigma-Aldrich (Merck, Rehovot, Israel) unless otherwise stated. SA conjugated to polyethylene glycol (PEG) (2 KDa) (SA-PEG) and SA-PEG with a carboxylic end group at the PEG terminus were custom-synthesized (Creative PEGWorks, Durham, NC). Glu-Urea-Lys with protecting groups of tert-Butyl esters was purchased from ABX advanced biochemical compounds (Radeberg, Germany). Cyanine7 with amine modification (Cy7-NH2) was purchased from Moshe Stauber Biotech Applications (Lod, Israel).

### Cell Cultures

Human prostate cancer cell lines LNCaP (overexpressing the PSMA receptor) and PC-3 (devoid of the PSMA receptor), human non-small cell lung cancer 1975 cell line, human embryonic kidney HEK-293 cell line, and normal human bronchial epithelial BEAS2B cell line were cultured in RPMI-1640 medium, supplemented with 10% fetal bovine serum (FBS), 2 mM glutamine, 100 μg/ml penicillin and streptomycin (Biological Industries, Beit-HaEmek, Israel). LNCaP cells growth medium was supplemented with 5 μg/ml insulin (Sigma-Aldrich, Merck, Rehovot, Israel). Neonatal foreskin fibroblast FSE cell line was cultured in DMEM medium, supplemented with 10% FBS, 2 mM glutamine, 100 μg/ml penicillin and streptomycin. Cells were incubated at 37° C. in a humidified atmosphere of 5% CO₂. The actual surface expression of the PSMA receptor by LNCaP and PC-3 cell lines was determined by immunohistochemistry, conducted by the department of pathology in Rambam Health Care Campus.

### Preparation of NLCs

Self-assembled NLCs were prepared by the surfactant-free nanoprecipitation method with several modifications for the stabilization of the nanocarriers system.

For the unloaded NPs, SA-PEG was dissolved in distilled water (DW) filtered through 0.22 μm syringe filter at 70° C. for the SA to be in its liquid state, as the melting point of SA was reported to be 69.3° C. A liquid lipid phase dissolved in ethanol (EtOH) was then added dropwise into the preheated aqueous phase under magnetic stirring of 600 rpm. The mixture was agitated at room temperature for 10 min to reach equilibrium. Different liquid lipids were studied including carvacrol, cinnamaldehyde, a-pinene, oleic acid (OA), orange oil, lemon oil, and jasmine oil. Samples were then transferred to a cold-water bath at 4° C. under stirring for another 10 min, for the solidification of the SA shell and in order to reach a final equilibrium.

### Synthesis of SA-PEG-Targeting Ligand (TL) and Preparation of Targeted NLCs

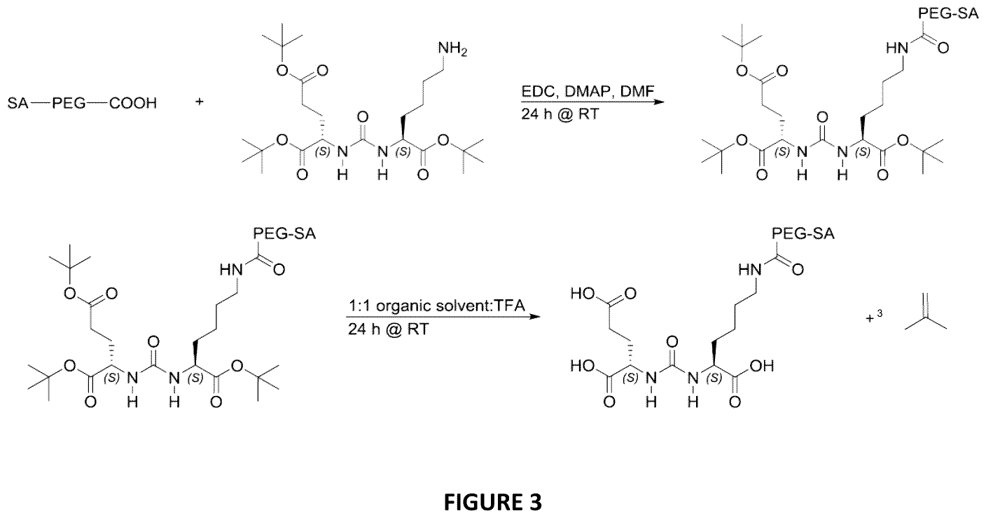

SA-PEG conjugated to PSMA targeting ligand (SA-PEG-TL) was synthesized by covalent coupling of SA-PEG-COOH with the amine group in the side chain of lysine in the PSMA TL Glu-Urea-Lys (FIG. 3). The carboxylic groups of Glu-Urea-Lys were masked by protecting groups of tert-Butyl esters. Briefly, SA-PEG-COOH (0.02 mmol), equal molar ratio of Glu-Urea-Lys TL (0.02 mmol), and the reagents 1-Ethyl-3-(3-dimethylaminopropyl)carbodiimide (EDC, 0.027 mmol), and 4-Dimethylaminopyridine (DMAP, 0.002 mmol) were dissolved in dimethylformamide (DMF, 1 ml), with continuous stirring at room temperature for 24 hr. DMF was evaporated and ¹H-NMR spectroscopy was conducted on a Bruker AVENCE II 400 MHz (¹H at 400 MHz) instrument in DMSO-d₆ as a solvent to validate the conjugation process. Elimination of impurities was achieved by dialysis (molecular weight cutoff of 500-100 Da, BDL (Beith Dekel) Ltd., Raanana, Israel) against DW for 24 hr. After conjugation and elimination of impurities, the SA-PEG-TL conjugation product was quantified using back titration. The excess of SA-PEG-COOH that did not react with the amine group on the PSMA TL was measured by titration with 0.01 M NaOH. The molarity of the SA-PEG-TL was calculated by the difference between the original molarity of SA-PEG-COOH and the molarity obtained by the acid-base titration. Thereafter, the PSMA TL protecting groups were removed under acidic conditions using trifluoroacetic acid (TFA) (FIG. 3) and the isobutylene by-product was evaporated together with the solvents. ¹H-NMR spectroscopy was conducted on a Bruker AVENCE 200 MHz (¹H at 200 MHz) instrument in DMSO-d₆ as a solvent to confirm the exposure of the carboxylic groups of the PSMA TL in the final SA-PEG-TL conjugation product.

For the preparation of targeted NLCs, SA-PEG-TL was added to the aqueous phase with SA-PEG at 70° C. OA dissolved in EtOH was then added dropwise into the preheated aqueous phase under magnetic stirring of 600 rpm at room temperature for 10 min. Samples were then transferred to a cold-water bath at 4° C. under stirring for another 10 min.

### Preparation of Drug-Loaded NPs

Drug-loaded NPs were prepared by the same method as described, with the addition of CTX dissolved in EtOH or ENZ dissolved in dimethyl sulfoxide (DMSO) in the OA liquid lipid phase. The mixture was added dropwise to the water phase, containing SA-PEG and SA-PEG-TL. Samples were stirred at room temperature for 10 min and transferred to 4° C. for another 10 min. Samples were then centrifuged at 10,000×g at 4° C. for 20 min to sediment the unbound excess drug aggregates.

### Particle Size Distribution and Zeta-Potential Analyses

The colloidal stability of the NLCs and the drug-loaded NPs were studied according to volume-weighted particles size distribution and zeta-potential using dynamic light scattering (DLS)/zeta-potential analyzer, Zetasizer Nano instrument (Malvern Instruments Ltd., Worcestershire, UK). Samples were prepared by the nanoprecipitation method as described above and measurements were carried out. Zeta-potential was calculated based on the Smoluchowski model. Measurements from two independent experiments, each performed in duplicates, are presented as means±SE.

### Analysis of Drug Loading Capacity and Encapsulation Efficiency

To quantify the amount of drug-loaded into the NPs, NLCs were prepared at increasing drug to SA-PEG molar ratios. The drug-loaded NPs were prepared as mentioned above and were centrifuged at 10,000×g at 4° C. for 20 min to sediment the unbound excess drug aggregates. Quantification of the encapsulated drug in the supernatant was performed by lyophilizing the supernatant and dissolving it in EtOH or DMSO to extract the CTX or ENZ, respectively, from the NLCs. The concentration of drugs was determined using reversed-phase HPLC (RP-HPLC) and analyzed using a linear calibration curve. RP-HPLC was conducted using a 4.6×250 mm C18 Kromasil RP-HPLC column and a UV detector at 234 nm or 280 nm for CTX or ENZ detection, respectively. For CTX quantification, the mobile phase consisted of acetonitrile (ACN) and water (50:50, v/v). For ENZ quantification, the mobile phase consisted of 55% ACN and 45% ammonium acetate buffer solution (10 mM, pH 5.0). The injection volume was 20 μl, at a flow rate of 1 ml/min. The column temperature was kept at 25° C., and the HPLC run was set for 25 min in the case of CTX or 15 min for ENZ with 6 min ACN rinse between runs.

Calculation of the loading capacity (LC, i.e., the mass ratio of drug to carrier) and encapsulation efficiency (EE) (i.e., the percent of encapsulated drug from the added drug) of the NPs was performed using Equations (1) and (2):

$$\\ \begin{matrix}LC {}^{} ( \frac{{mg}_{drug}}{{g}_{NLC}} ) = \frac{{W}_{ED}}{{W}_{SA - PEG}} & ( 1 )\end{matrix}$$

$$\\ \begin{matrix}EE ( \% ) = \frac{{W}_{ED}}{{W}_{TD}} * 100 \% & ( 2 )\end{matrix}$$

W_{ED} is the amount of encapsulated drug; W_{SA-PEG} is the total amount of SA-PEG in the sample; and W_{TD} is the total amount of drug in the sample.
Results from two independent experiments, each performed in duplicates, are presented as means±SE.

### Cryogenic-Transmission Electron Microscopy Imaging (Cryo-TEM)

Cryo-TEM (Philips CM120 microscope) analysis was used for the imaging of NLCs, NPs loaded with 0.6:1 CTX:SA-PEG molar ratio and NPs loaded with 0.4:1 ENZ:SA-PEG molar ratio. Samples were prepared as described and images were taken at 4° C. Gatan Multi Scan 791 cooled CCD camera was used to acquire the images, using the Digital Micrograph 3.1 software package. NPs size was determined using ImageJ software.

### Kinetic Profile of In Vitro Drug Release

The kinetics of in vitro release of drugs from the loaded NPs and free drugs were studied by the dialysis method. One (1) ml of drug-loaded NPs or free drugs (both systems in DW) were placed in dialysis bags (molecular weight cutoff of 3.5 kDa, Sigma-Aldrich, Merck, Rehovot, Israel) and incubated in 30 ml volume of plasma simulating buffer comprising phosphate-buffered saline (PBS, 10 mM, pH 7.4) containing 0.1% tween 80 for 72 hr, at 37° C., with continuous gentle agitation. At defined time intervals (0, 2, 4, 6, 10, 23, 29, 36, 47, 58, 72 hr), the dialysate was collected and replaced by the same volume of fresh release buffer. To calculate the cumulative release of drugs, the dialysates from each time point were freeze-dried, dissolved in EtOH or DMSO for CTX or ENZ extraction, respectively, filtered through 0.45 μm membrane, and quantified using RP-HPLC, as described above. The results are presented as means of two experiments±SE.

### Determination of NPs Concentration by Nanosight

NPs concentration was determined using Nanosight instrument (NS300, Malvern Instruments Ltd., Worcestershire, UK). Samples were prepared as described, diluted with DW, and measured at 25° C. Results are presented as means of two independent experiments±SE.

### Characterization of NPs Specificity by Confocal Laser Microscopy

Fluorescent NPs were formed as described, with the addition of SA-PEG conjugated to the fluorophore Cy7 (SA-PEG-Cy7) to the aqueous phase during NPs preparation. The conjugation between Cy7-NH₂ and SA-PEG-COOH was performed using the EDC and N-hydroxysuccinimide (NHS) conjugation method as previously described (77). SA-PEG-COOH (0.0125 mmol) was incubated with an excess of EDC and NHS (at a 1:2 EDC:NHS molar ratio) for 15 min at room temperature with gentle shaking. The resulting NHS-activated SA-PEG was covalently linked to Cy7-NH₂ (0.0125 mmol). The sample was allowed to react for 2 hr with constant mixing at room temperature, and the final conjugate was dialyzed against DW (molecular weight cutoff of 3.5 kDa, Sigma-Aldrich, Merck, Rehovot, Israel) for 24 hr with 5 replacements of the dialysate to remove unreacted fluorophore.

Selective internalization of Cy7-labeled NPs was studied using confocal fluorescence microscopy (inverted confocal microscope, Zeiss LSM 710). Prior to the experiments, cells were seeded on μ-slides VI 0.4 (Ibidi, Martinsried, Germany) at 50% confluence and incubated overnight. Cells were then washed with PBS and incubated with 1 μg/ml Hoechst 33342 in growth medium for 10 min to achieve nuclear DNA staining. Thereafter, cells were incubated with different fluorescent NPs.

For selective internalization of targeted NPs as a function of PSMA TL concentration, LNCaP target cells were incubated for 2 hr at 37° C., with non-targeted NPs and NPs decorated with increasing TL concentrations of 15 nM, 30 nM, and 80 nM (diluted 1:400 (v/v) in FBS-free medium). Thereafter, cells were washed twice with PBS to remove free fluorescent NPs. Two fluorescence channels were used during image capture: (1) Blue for the viable DNA-dye Hoechst 33342 (Excitation/Emission: 350/461 nm); and (2) Deep red for the Cy7-labeled NPs (Excitation/Emission: 720/750 nm).

For specific internalization study, LNCaP and PC-3 cell lines were incubated with non-targeted fluorescent NPs or 30 nM TL decorated fluorescent NPs (diluted 1:400 (v/v) in FBS-free medium) for 2 hr at 37° C. In addition, 1975 human non-small cell lung cancer, HEK293, BEAS2B, and FSE cells, were incubated with targeted fluorescent NPs (diluted 1:400 (v/v) in FBS-free medium) for 2 hr at 37° C. Then, cells were washed twice with PBS to remove free fluorescent NPs, and confocal microscope images were captured with the same fluorescence channels as mentioned above.

For characterization of active internalization of NPs, LNCaP cells were incubated with a serum-free medium containing targeted NPs (diluted 1:400 (v/v)) for 1 hr, at two different temperatures: 4° C. and 37° C. Following incubation, cells were washed twice with PBS and the cellular fluorescence pattern was examined. Images were analyzed with IMARIS software.

### Growth Inhibition Assays

The selective growth inhibition of CTX-loaded NPs was studied in LNCaP target cells and PC-3 non-target cells, using an XTT-based colorimetric cell proliferation kit as previously described. The preparation of CTX-loaded NPs was performed as described and had undergone 24 hr dialysis before cell exposure, to simulate the conditions in the human body. The dialysis was conducted as described in the “Kinetic profile of in vitro drug release” in the materials and methods section, but with the use of 10% FBS to capture the released CTX. Prior to the experiment, 96-well plates were coated with poly-L-Lysine before cell plating. LNCaP and PC-3 cells were seeded in 96-well plates at 5×10⁴ and 2.5×10⁴ cells/ml, respectively, and incubated overnight to allow for cell attachment. Following exposure of cells to CTX-loaded NPs at increasing CTX concentrations of 0.001-100 nM for 24 hr, cells were incubated for additional 72 hr with fresh growth medium. Cellular growth inhibition was determined by adding the XTT reagent. Data were plotted using a nonlinear curve fitting of a sigmoidal model (Hill1) with OriginPro 9.0 to obtain a dose-response curve according to Equation (3):

$$\\ \begin{matrix}P = {P}_{∞} + ( {P}_{0} - {P}_{∞} ) \times ( \frac{{[ D ]}^{n}}{{IC}_{50}^{{}^{} n} + {[ D ]}^{n}} ) & ( 3 )\end{matrix}$$

P stands for the percentage of live cells; P_{∞} represents the minimal percent of live cells at infinite drug concentration; P₀ is the maximal percent of surviving cells in the absence of drug (100%=control); [D] stands for the drug concentration; IC₅₀ stands for the drug concentration exerting 50% inhibition of cell growth; n indicates the abruptness of the dose-response curve. Cells grown with free CTX at the same concentrations or medium with the addition of non-encapsulating NLCs served as the controls.

Results shown are means of two independent experiments, each performed in triplicates±SE.

### Example 1

### Self-Assembly of the Delivery System

NLCs are typically composed of a mixture of solid and liquid lipids at different ratios, designed to obtain stable core-shell structured spherical particles. Different formulations were examined, and a mixture of 60% SA-PEG and 40% liquid lipid was selected for further research. Self-assembled NLCs were prepared as described in the “Preparation of NLCs” (materials and methods section).

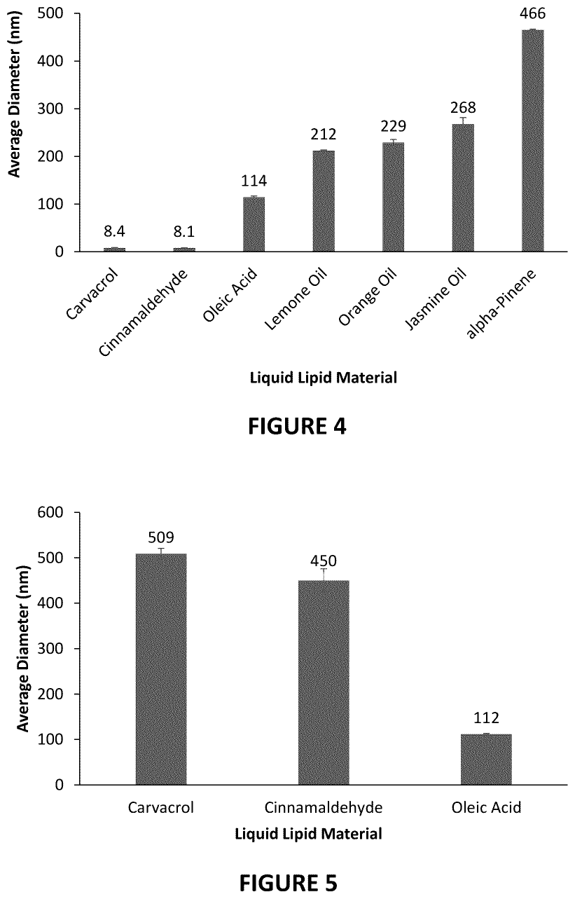

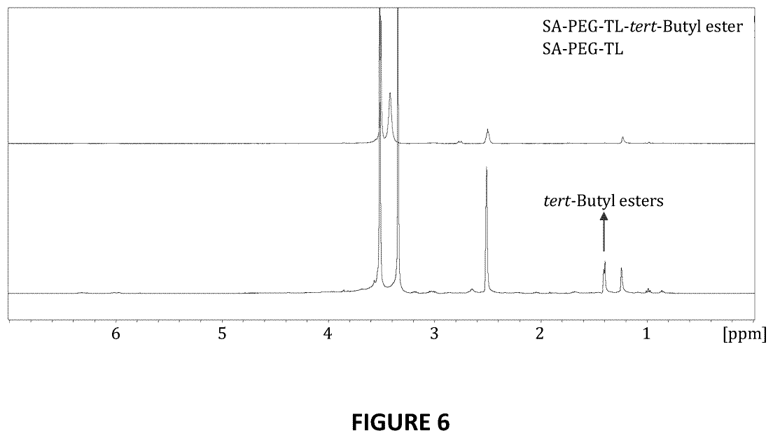

Various liquid lipids were studied according to the nanoparticles' size distribution, as an assessment of the compatibility between the crystalline shell created by solidifying SA and the liquid lipid core (FIG. 4).

To form a stable colloidal system of NPs suitable for effective accumulation in the TME and internalization to target cancer cells via endocytosis, the inventors aimed at forming NPs with a mean diameter of 50-200 nm and a zeta-potential below −30 mV. NPs with 40% carvacrol, cinnamaldehyde, and oleic acid displayed a monomodal distribution with a mean size of less than 200 nm and were further studied for their size distribution upon ENZ encapsulation at a 0.4:1 ENZ:SA-PEG molar ratio (Error! Reference source not found.). A large increase in NP size was observed with carvacrol and cinnamaldehyde (8.4±0.1 nm to 509±12 nm and 8.1±0.1 nm to 450±26 nm, respectively), whereas NPs with OA demonstrated robust size and were therefore selected as the final formulation for further experiments.

To generate NPs that will selectively target PC cells, SA-PEG was conjugated via an amide linkage to the PSMA TL Glu-Urea-Lys. SA-PEG with a carboxylic end group at the PEG terminus and the amine group in the Lysine side chain of the Glu-Urea-Lys TL with protecting groups of tent-Butyl esters, were conjugated for the synthesis of SA-PEG-TL (Error! Reference source not found.). Following conjugation and elimination of unwanted impurities, the protecting groups were removed to reveal the three carboxylic groups, constituting the binding motif to the PSMA receptor (Error! Reference source not found.). NLCs with the incorporation of the PSMA TL (Targeted-NLCs) were slightly enlarged to an average diameter of 129±3 nm, and their zeta-potential was −36.3±0.3 mV. This negative surface charge of NPs has been achieved thanks to the reduction of the carboxylic protecting groups of tert-Butyl esters, as shown in the H-NMR spectroscopy (Error! Reference source not found.), thus exposing the TL's negative charge at physiological pH.

These results are an indication of an accomplishment of a stable, robust, and efficient nanocarrier system that was subjected to further experiments.

### Example 2

### Physiochemical Characterization of NPs

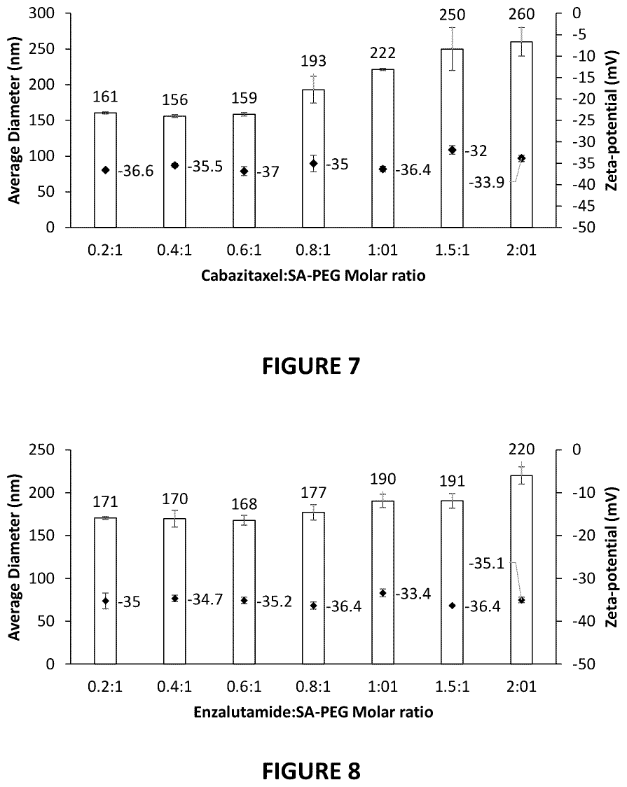

Size distribution and zeta-potential of targeted-NLCs were determined at increasing drug to SA-PEG molar ratios using DLS/zeta-potential analyzer. CTX or ENZ was dissolved in the liquid lipid phase together with the OA and loaded into the targeted NLCs by physical encapsulation. Following centrifugal sedimentation of the unbound excess drug, CTX- and ENZ-loaded NPs were studied for size distribution and had displayed a monomodal distribution (FIGS. 7 and 8, respectively).

Expectedly, at increasing drug to SA-PEG molar ratios, there was an increase in the average diameter of NPs. This is due to the encapsulation of molecules that are characterized by a hydrophobic taxadiene core as in the case of CTX or multiple benzamides as in ENZ. As the drug:SA-PEG ratio increases, the volume:surface ratio increases and thus the NPs average diameter increases. In contrast, the zeta-potential of NPs remained negative and in the range of −35±3 mV. As the inventors aimed to obtain NPs with an average size of less than 200 nm, CTX:SA-PEG molar ratio of 0.2-0.6:1 and ENZ:SA-PEG molar ratio of 0.2-0.8:1 appeared to be suitable, and the drug loading capacity was hence studied.

### Example 3

### Drug Loading Capacity (LC) and Encapsulation Efficiency (EE)

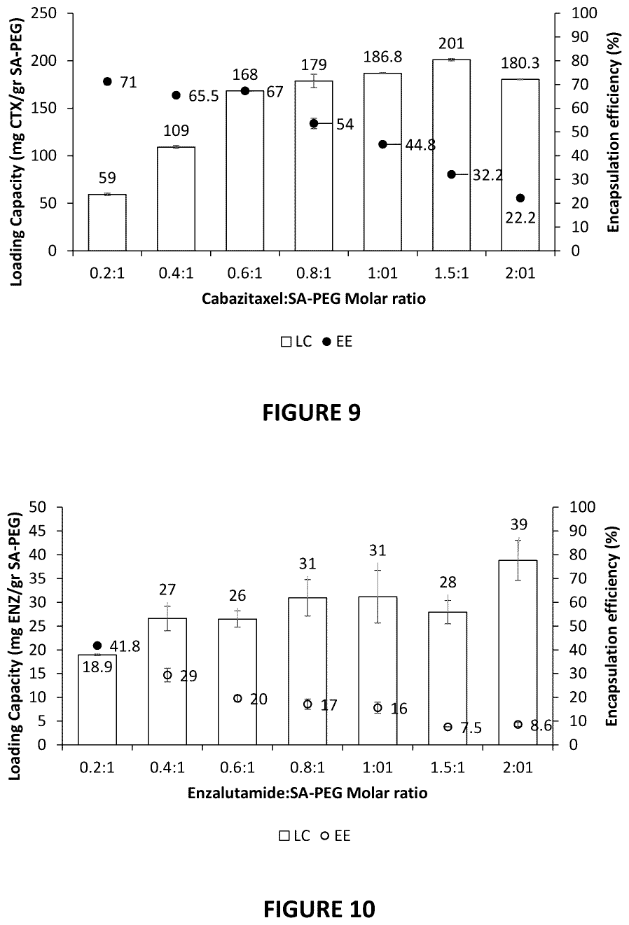

The LC and EE of CTX and ENZ at increasing drug to SA-PEG molar ratios were determined by centrifugal sedimentation of unbound excess drug and quantification of the encapsulated drug in the supernatant by RP-HPLC (FIGS. 9 and 10, respectively). Expectedly, at increasing drug to SA-PEG molar ratios, the EE decreased, whereas the LC increased. It is evident from FIG. 9 that increased concentrations of CTX led to a significant increase in LC, up to 0.6:1 CTX to SA-PEG molar ratio. However, at higher drug concentrations, the increase in LC became more moderate. These results are consistent with the DLS data showing a major increase in the average diameter of NPs between 0.6:1 to 0.8:1 CTX:SA-PEG molar ratio (159±3 nm and 193±19 nm, respectively). Taken collectively, the formulation which displayed the optimal relation between LC, EE, and particle size was 0.6:1 CTX:SA-PEG, with an average diameter of 159±3 nm, LC of 168±3 mg CTX/g SA-PEG, and EE of 67±1%.

In the case of ENZ encapsulation, the EE and LC values were much lower than with CTX encapsulation. This result is presumably because of lower compatibility between the OA liquid core, that constitutes the drug dispersion component, and the ENZ that was initially dissolved in DMSO. The analysis of DLS measurements revealed NPs with the same size range up to a molar ratio of 0.8:1 ENZ:SA-PEG and a major increase in LC between 0.2:1 to 0.4:1 ENZ-SA-PEG (18.9±0.2 and 27±3 (mg ENZ/g SA-PEG)). Hence, the formulation that was selected for subsequent experiments is 0.4:1 ENZ:SA-PEG molar ratio, with an average diameter of 170±10 nm, LC of 27±3 mg ENZ/g SA-PEG, and EE of 29±3%.

### Example 4

### The Morphology of Drug-Loaded NPs

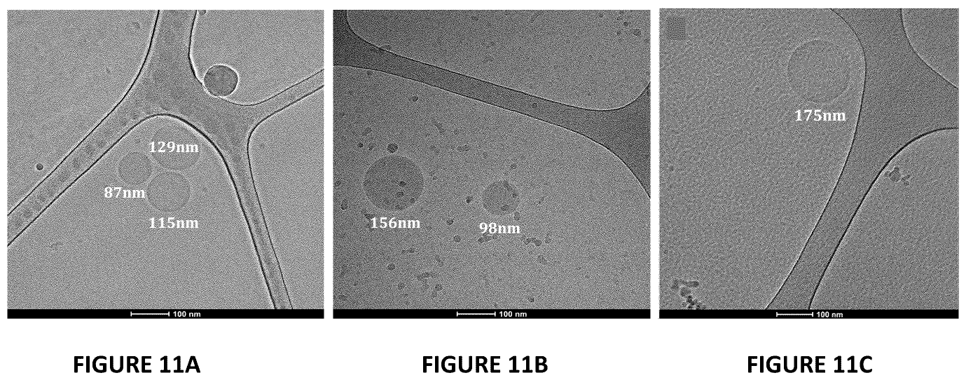

The morphology of selected NPs was examined by analyzing cryogenic transmission electron microscopy (Cryo-TEM) images (FIG. 11). NPs loaded with CTX (FIG. 11A), and NPs loaded with ENZ (FIG. 11B) appear as spherical particles with an average diameter, which is in agreement with the DLS measurements. As it was previously found that spherical particles are able to undergo faster internalization than non-spherical NPs, this result contributes to the efficiency of the delivery system. Additionally, non-encapsulated NLCs (FIG. 11C) displayed a similar morphology, supporting the incorporation of drugs into the NLCs nanocapsules.

### Example 5

In Vitro Release Profile of CTX and ENZ from the NPs

The kinetics of in vitro release of CTX and ENZ from NPs were assessed by dialysis in a large volume of a plasma-simulating buffer for 72 hr at 37° C., with continuous mixing. The plasma-simulating buffer was composed of PBS at pH 7.4 and contained 0.1% wt Tween 80 to capture the released hydrophobic drug, hence mimicking drug capture by human serum albumin in the plasma. The above conditions were meant to simulate the systemic circulation of NPs in the blood over 24-48 hr, assuming this is a typical time for circulating NPs to accumulate in the tumor via the EPR effect. At defined time intervals, the dialysate was replaced with a fresh buffer, and the drug was quantified using RP-HPLC. The complete replacement of fresh release buffer caused a stronger release driving force and constituted stringent release conditions.

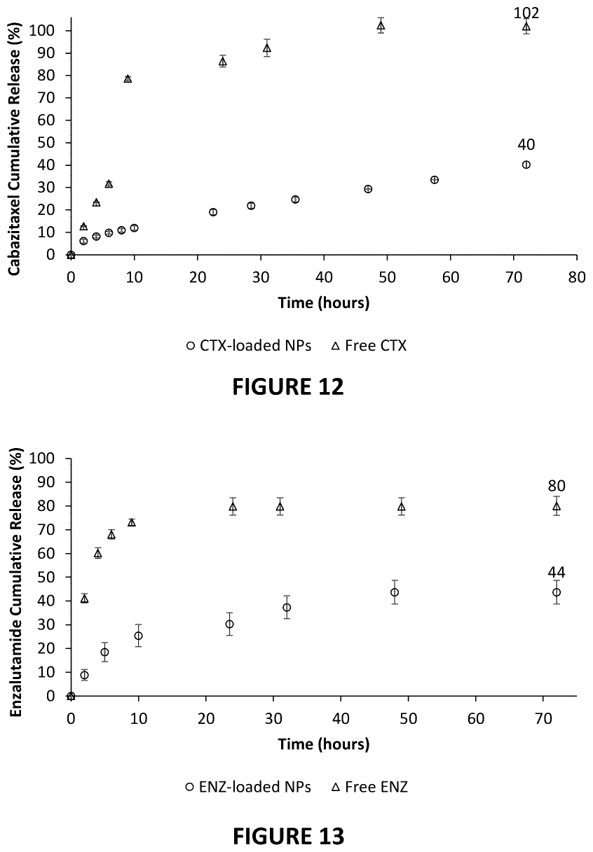

The in vitro release profile of CTX and ENZ from NPs showed substantial drug retention, as opposed to a burst release of the unencapsulated drug during the first 10 hr (FIGS. 12 and 13, respectively). These results are affected by the ratio between the liquid and solid lipids in the NPs formulation. As previously reported, NLCs' solid lipids form a crystalline shell that acts as a barrier, and the liquid lipids form a cohesive hydrophobic core with strong interaction with the drugs. Thus, a substantial reduction in drug expulsion from the NPs was observed. However, the initial release of drug from NPs, probably of the drug that was dispersed in the outer shell of NPs, was slightly more rapid. This is due to the phase separation that occurs during the crystallization of the solid lipids shell, as was previously found for NLCs.

### Example 6

### Selective Targeting and Internalization of NPs Into LNCaP Target Cells

The selective internalization of targeted NPs labelled with the deep red fluorescent dye, Cy7, was studied using confocal laser microscopy. The cell lines that were examined were LNCaP, PC cells overexpressing PSMA that were used as the target cells; PC-3, rare PC cells lacking the PSMA expression; non-small cell lung cancer 1975 cells; human embryonic kidney HEK-293 cells; normal human bronchial epithelial BEAS2B; and neonatal foreskin fibroblast FSE cells.

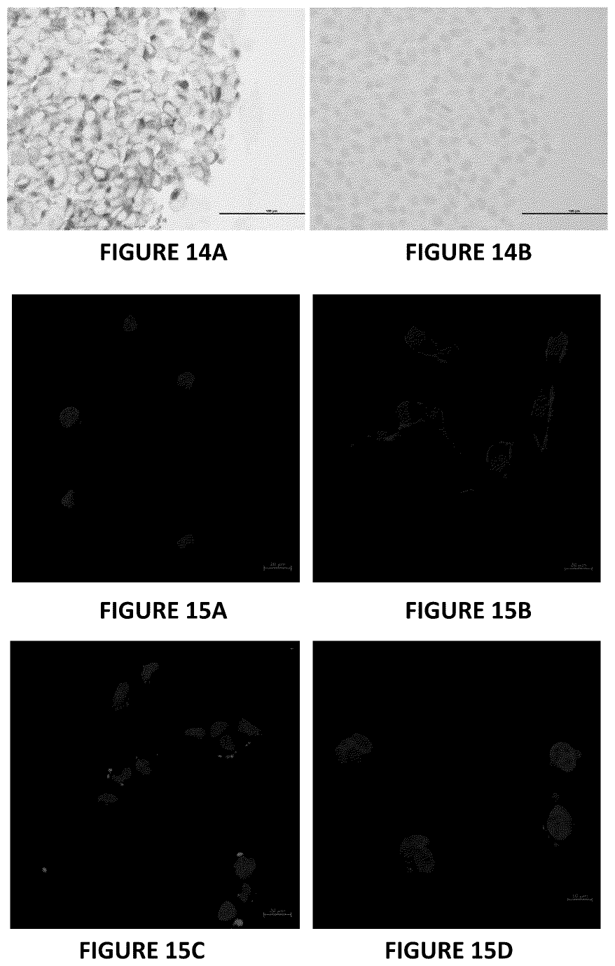

First, the actual surface expression of PSMA by the above PC cell lines was determined by immunohistochemistry, conducted by the department of pathology in Rambam Health Care Campus. Expectedly, LNCaP cells displayed PSMA overexpression (FIG. 14A) whereas no expression was found in PC-3 cells (FIG. 14B). These results are shown by the brown staining of the PSMA receptor that was largely confined to the plasma membrane of LNCaP cells.

The optimal amount of TL conjugated to the NPs was examined by evaluation of the fluorescence signal in LNCaP target cells (FIG. 15). The NPs were decorated with TL concentrations of 15 nM, 30 nM, and 80 nM or with no TL at all. The inventors found that the internalization of NPs into LNCaP target cells is dependent on the concentration of TL decorating the NPs surface. Following 2 hr incubation with NPs decorated with increasing TL concentrations at 37° C., LNCaP cells displayed maximal internalization with 30 nM TL decoration. Possible reasons for the result that optimal NPs internalization occurred at 30 nM TL decoration are: a) A large number of TLs on the NPs surface may facilitate over-binding of a single NP to multiple PSMA receptors thereby impairing proper internalization; b) The large number of TLs sterically hindered these ligands from binding to the cell membrane PSMA receptors; c) The negative surface charge of the NPs caused by the large number of dissociated carboxylic groups on the PSMA TLs, resulted in an extensive repulsion between NPs and the negatively charged plasma membrane.

The observation of the complete lack of internalization upon exposure of target cells to non-targeted NPs, confirmed that the targeting properties were specifically attributed to the PSMA TL, Glu-Urea-Lys. To conclude, the targeted NPs selected for further studies were those decorated with 30 nM PSMA TL.

Determination of NPs concentration by Nanosight enabled to calculate of the number of TL molecules on the NPs surface. NPs concentration was found to be 0.28894±0.00007 nM, thus decorating of NPs with 30 nM TL led to ˜100 TLs per particle.

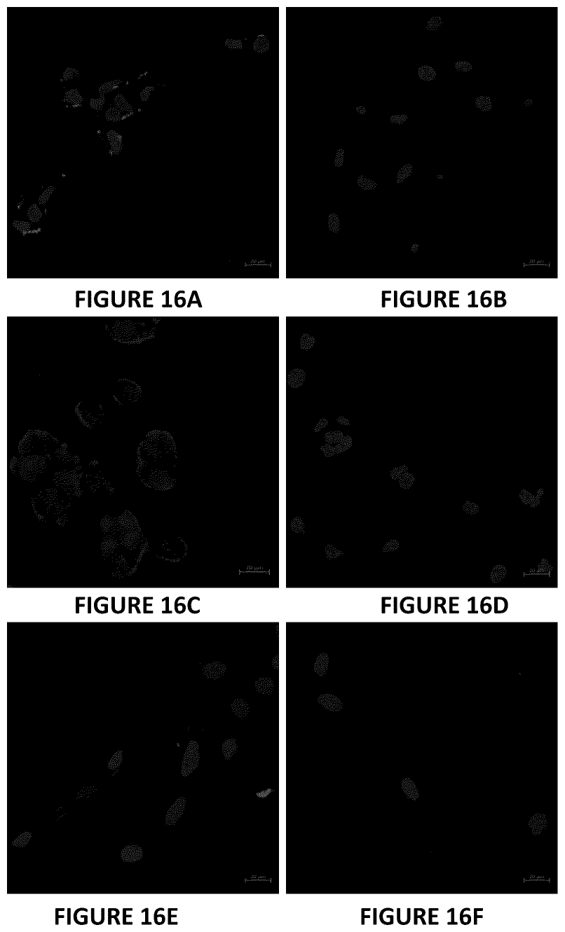

To ensure the specific internalization of targeted NPs to LNCaP target cells via the PSMA receptor, different cell lines were examined including PC-3, 1975, HEK-293, BEAS2B, and FSE cell lines (FIG. 16).

Neither binding nor internalization were observed in non-target cells, demonstrating absolute specificity and enhanced internalization of NPs by PSMA expressing PC cells. Moreover, the accumulation of targeted NPs within LNCaP cells has shown perinuclear punctate staining. This indicates internalization by pit formation on the cells' membrane and intracellular accumulation in endolysosomes.

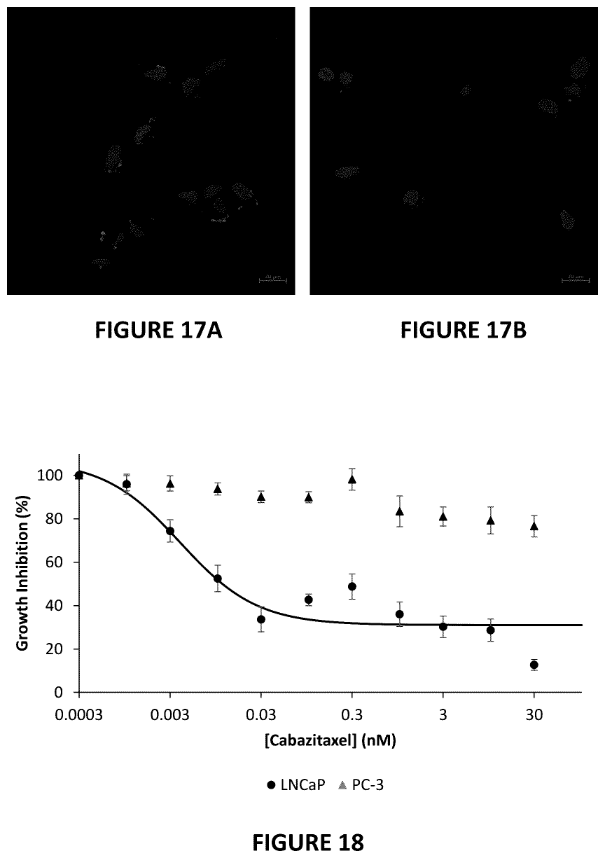

For the characterization of active internalization by endocytosis, cells were incubated at two different temperatures: 4° C. and 37° C. As endocytosis is an energy-dependent process, the inventors assumed there will be higher accumulation at 37° C., while at 4° C. only binding to the PSMA receptor may occur. Confocal microscopy analysis (FIG. 17) revealed that the uptake of targeted NPs was based on endocytic processes, with 17 fluorescent dots per target cell and an average intensity of 120±60 (A.U) at 37° C. compared to 4 dots/cell and average intensity of 63±30 (A.U) at 4° C. It should be noted that cellular processes other than endocytosis, such as diffusion, are also impeded at low temperatures and it is possible that TL binding to the PSMA receptor encouraged the diffusion of the NPs into the cell.

### Example 7

### Selective Growth Inhibition of CTX-Loaded NPs In Vitro

Cytotoxicity assays were performed using an XTT-based colorimetric cell proliferation kit with the PSMA-positive PC cell line (LNCaP), and PSMA-negative PC cell line (PC-3).

It is evident from FIG. 18 that CTX-loaded NPs displayed a remarkable dose-dependent growth inhibitory effect on target LNCaP cells which overexpress PSMA, with a half-maximal inhibitory concentration (IC₅₀) value of 0.004±0.002 nM. In contrast, these NPs did not exhibit any substantial growth inhibitory effect on PC-3 which lack PSMA expression.

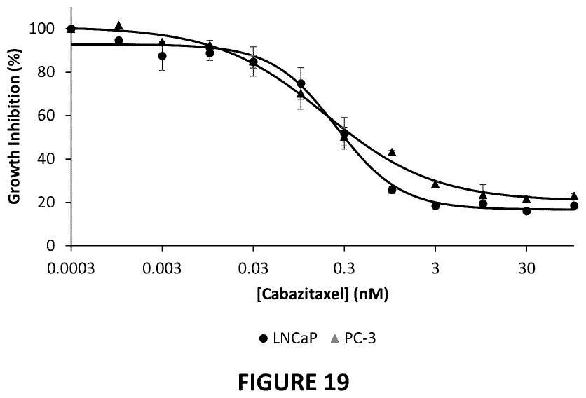

Cells exposed to free CTX in growth medium for 72 hr served as control and similar IC₅₀ values of 0.25±0.04 nM and 0.18±0.04 nM were obtained with LNCaP and PC-3 cells, respectively (FIG. 19). The ratio of 62.5-fold between the IC₅₀ values of free CTX and CTX-loaded NPs towards target LNCaP cells can be explained by the different exposure times and the targeting ability of the CTX-loaded NPs. The active targeting of CTX-loaded NPs enables an accumulation of NPs in target PC cells, and thus, a more potent anti-tumor activity. As a result, even in shorter exposure times (24 hr, compared to 72 hr of free CTX), the cytotoxic effect of the NPs was more significant against LNCaP target cells, as evident from the lower IC₅₀ value. Moreover, it has been reported that the IC₅₀ of free Cabazitaxel exposure for 48 hr towards LNCaP cells is 2.6-3.1 nM, which supports this conclusion.

It should be mentioned that non-encapsulating NLCs were found to be non-toxic to cells.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A conjugate comprising a fatty acid derivative covalently bound to a prostate specific membrane antigen (PSMA) ligand, wherein said covalently bound is via a linker; wherein said PSMA ligand is represented by Formula 1: 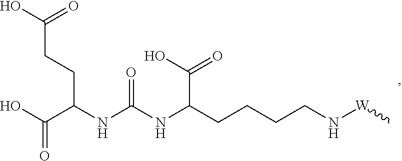
including any salt, and any stereoisomer thereof; wherein W is a spacer, or is absent; and wherein the wavy bond represents an attachment point to the linker;
wherein the fatty acid derivative is represented by Formula: 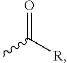
wherein R represents a C5-C30 alkyl, a C5-C30 alkenyl, or a C5-C30 alkynyl, wherein each of the alkyl, alkenyl and alkynyl is optionally substituted; and wherein the wavy bond represents an attachment point to the liker.

2. The conjugate of claim 1, wherein W is absent, and wherein a first portion of said linker is covalently bound to a carbonyl group of the fatty acid derivative , and a second portion of said linker is covalently bound to an c-amine of the lysine of said PSMA ligand.

3. The conjugate of claim 1, wherein said fatty acid derivative is a C10-C30 fatty acid derivative; and wherein said linker comprises PEG.

4. (canceled)

5. The conjugate of claim 1, wherein the linker has a molecular weight (MW) between 1,000 and 5,000 Dalton (Da).

6. The conjugate of claim 2, wherein the ε-amine of the lysine is bound to the linker via an amide bond.

7. The conjugate of claim 1, wherein the conjugate is represented by Formula 2: 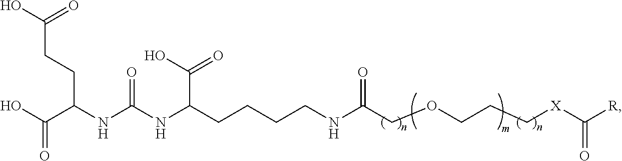
including any salt, and any stereoisomer thereof; wherein:
R is selected from a C10-C30 alkyl, a C10-C30 alkenyl, and a C10-C30 alkynyl;
X represents O, NH, or S;
each n is independently 0, or between 1 and 10; and
m is between 2 and 100.

8. A composition comprising a plurality of nanoparticles, wherein each of said plurality of nanoparticles comprises a core and a shell, wherein:
said shell comprises the conjugate of claim 1; and
said core comprises a liquid oil.

9. The composition of claim 8, further comprising a PEG-ylated fatty acid and an active agent; optionally wherein a weight ratio between the liquid oil and the PEG-ylated fatty acid is between about 1:1 and about 1:3.

10. (canceled)

11. The composition of claim 9, wherein said pharmaceutically active agent is an anti-cancer agent; and wherein said anti-cancer agent is an oil-soluble compound.

12. The composition of claim 8, wherein said plurality of nanoparticles is characterized by: (i) an average particle size in a range of between about 30 and about 300 nm; (ii) a negative zeta potential between 0.5 and 50 mV.

13. (canceled)

14. (canceled)

15. The composition of claim 12, wherein said average particle size is between about 50 and about 200 nm, and wherein a molar ratio between the pharmaceutically active agent and the PEG-ylated fatty acid is between about 0.1:1 and about 1:1; optionally wherein said liquid oil is a fatty acid; optionally wherein said composition further comprises an aqueous solution, wherein said plurality of nanoparticles is dispersed within said aqueous solution.

16. (canceled)

17. (canceled)

18. A pharmaceutical composition comprising the composition of claim 8, and a pharmaceutically acceptable carrier.

19.-26. (canceled)

27. A method for treating a PSMA-related disease or disorder in a subject in need thereof, the method comprising administering to said subject a therapeutically effective amount of the pharmaceutical composition of claim 18, thereby treating the PSMA-related disease or disorder in a subject.

28. The method of claim 27, wherein said PSMA-related disease or disorder is a cell-proliferation related disease.

29. The method of claim 28, wherein said cell-proliferation related disease is cancer.

30. The method of claim 29, wherein said cancer is prostate cancer.

31. The method of claim 30, wherein at least one cell of said prostate cancer is characterized by increased expression, abundance, or both, of PSMA transcript, protein product thereof, or both, compared to a control.

32. The method of claim 27, further comprising a step preceding said administering, comprising determining expression, abundance, or both, of PSMA transcript, protein product thereof, or both, in a sample derived or obtained from said subject, wherein an expression, abundance, or both, of said PSMA transcript, protein product thereof, or both, being above a predetermined threshold, is indicative of said being suitable for said administering.

33. The method of claim 32, wherein said sample comprises at least one cell of said prostate cancer.

34. The method of claim 27, wherein said treating comprises: reducing the number of proliferating cells of said cancer, reducing the rate of cell proliferation of cells of said cancer, reducing the survival or viability of cells of said cancer, or any combination thereof.
